(19) United States
(12) Patent Application Publication
(10) Pub. No.: US 2018/0021303 A1
(43) Pub. Date: Jan. 25, 2018

(54) BKCA CHANNEL ACTIVATOR FOR TREATING MUSCULAR DISORDER

(71) Applicant: Canbex Therapeutics Limited, London (GB)

(72) Inventors: David Selwood, Welwyn Garden City (GB); David Baker, London (GB)

(21) Appl. No.: 15/550,594

(22) PCT Filed: Feb. 12, 2016
(86) PCT No.: PCT/GB2016/050353

§ 371 Date: Aug. 11, 2017

(30) Foreign Application Priority Data
Feb. 13, 2015 (GB) 1502412.8

(51) Int. Cl. A61K 31/404; A61K 31/407; A61K 31/41; A61K 31/4184; A61K 31/4245

(52) U.S. Cl. CPC A61K 31/404; A61K 31/4184; A61K 31/4245; A61K 31/41; A61K 31/407

(57) ABSTRACT

The present invention relates to BKCa activators for use in the treatment of a muscular disorder, or for controlling spasticity or tremors, for example, spasticity in MS.

The present invention relates to compounds useful in the treatment of muscular disorders, or for controlling spasticity or tremors.

### BACKGROUND TO THE INVENTION

Spasticity is a motor disorder clinically defined as a velocity-dependent increase in muscle tone resulting from hyperexcitable stretch reflexes, spasms and hypersensitivity to normally innocuous sensory stimulations. The intermittent or sustained involuntary muscle hyperactivity that characterises spasticity is associated with upper motor neurone lesions that can be located anywhere along the path of the corticospinal (pyramidal) tracts. This includes the motor pathways of the cortex, basal ganglia, thalamus, cerebellum, brainstem or spinal cord.

### Mechanisms of Spasticity

The aetiology of spasticity in MS has been relatively little studied. This is in contrast to spasticity caused by spinal cord injury, where the control of chloride homeostasis has recently been invoked as a key mechanism mediating spasticity (Boulenguez P., Liabeuf S., Bos R., Bras H., Jean-Xavier C., Brocard C., Stil A., Darbon P., Cattaert D., Delpire E., Marsala M., Vinay L., (2010), Nat Med 16:302-307). Previous studies have suggested that a complex system of ion channels and transporters controls neuronal excitability versus inhibitory signalling in the spinal cord. GABA is the major inhibitory transmitter in the spinal cord and the GABA_{A} agonist Baclofen is a treatment for spasticity, glycine is also an inhibitory neurotransmitter. Both GABA and glycine act at chloride ion channels. Low intracellular chloride ion concentrations were thought to mediate inhibitory signalling and concentrations of chloride are maintained at a low level by the potassium/chloride ion transporter KCC2. At these low concentrations of chloride, opening of GABA_{A} channels and glycine channels were understood to increase chloride concentrations and cause a hyperpolarising current. Following spinal cord injury, it was understood that KCC2 becomes downregulated, chloride levels increase and glycine mediates depolarization. While many details remain to be elucidated, the overall effect is to diminish the inhibitory signal to the muscles leading to excessive excitability, contraction and spasticity. As such, deficiency in the glycine receptor in mice leads to neurological abnormalities in early juvenile life in a mouse called the Spastic mouse (von Wegerer J., Becker K., Glockenhammer D., Becker C. M., Zeilhofer H. U., Swandulla D., (2003), Neurosci Lett 345:45-48).

To study MS related spasticity, a chronic relapsing EAE model has been developed (Baker D., Pryce G., Croxford J. L., Brown P., Pertwee R. G., Huffman J. W., Layward L. (2000), Nature 404:84-87). Efficacy in this system was demonstrated by Baclofen, endocannabinoids and cannabinoids, and has been translated to the treatment of MS. More recent evidence points to modulatory sites on glycine channels (GlyRs) for endocannabinoids (Yevenes G. E., Zeilhofer H. U. (2011), PLoS One 6:e23886) and these may contribute to the effect on spasticity. The functions of glycine signalling have been primarily studied in pain, however it has been shown that methanandamide, the synthetic analogue of the endogenous cannabinoid anandamide, can alleviate spasticity in the chronic relapsing EAE model (Brooks J. W., Pryce G., Bisogno T., Jaggar S. I., Hankey D. J., Brown P., Bridges D., Ledent C., Bifulco M., Rice A. S., Di Marzo V., Baker D. (2002), Eur J Pharmacol 439:83-92; Baker D., Pryce G., Croxford J. L., Brown P., Pertwee R. G., Huffman J. W., Layward L. (2000), Nature 404:84-87).

With regard to the involvement of glycine in spasticity mechanisms, mutations in the glycine receptor demonstrate an important role in the control of muscle tone as shown by studies in mouse strains (Oscillator, Spasmodic and Spastic). The archetypal glycine antagonist, strychnine causes severe muscle cramps. A hyperekplexic response (an exaggerated startle response to tactile or acoustic stimuli) is observed in humans with similar mutations. Similar responses have now been shown in humans with mutations in the glycine transporter GlyT2a (Rees M. I , Harvey K., Pearce B. R., Chung S. K., Duguid I. C., Thomas P., Beatty S., Graham G. E., Armstrong L., Shiang R., Abbott K. J., Zuberi S. M., Stephenson J. B., Owen M. J., Tijssen M. A., van den Maagdenberg A. M., Smart T. G., Supplisson S,. Harvey R. J. (2006), Nat Genet 38:801-806).

The present invention seeks to provide a new class of compounds having therapeutic applications in the treatment of muscular disorders, particularly for controlling spasticity and/or tremors.

### STATEMENT OF INVENTION

A first aspect of the invention relates to a BKCa channel activator for use in treating a muscular disorder, or for use in treating or controlling spasticity or tremors. Studies by the applicant have demonstrated that the BKCa channel activator BMS 204352 is effective in a mouse model of spasticity in multiple sclerosis. Moreover, studies with other structurally unrelated compounds, including NS 1619 and NS 11021, have shown that this appears to be a mechanism common to the class of BKCa activators in general.

### DETAILED DESCRIPTION

### BKCa Activators

BK channels (BKCa channels, Maxi-K channels, large-conductance Ca²⁺-activated K⁺ channels, KCal.1, KCNMA1, Slol) are expressed in a wide variety of cells including most neurons, muscle, epithelia, endothelia and endocrine cells. The pore-forming α-subunit of the BK channels is coded for by the single gene KCNMA1, but the diversity of the BK channels is largely due to a number of C-terminal splice variants. The diversity is further increased by the presence of several accessory β-subunits, which modulate the function of the channels and are coded for by the KCNMB1-4 genes (Salkoff L. et al Nat Rev, 2006, 7(12), 921-931; Nourian, Z., M. Li, M. D. Leo, J. H. Jaggar, A. P. Braun and M. A. Hill (2014), “Large conductance Ca²⁺-activated K⁺ channel (BKCa) alphα-subunit splice variants in resistance arteries from rat cerebral and skeletal muscle vasculature,” PLoS One 9(6): e98863).

The BK channel complex is composed of 4 α-subunits, each spanning the membrane 7 times, plus 1-4 β-subunits (β1-β4), each spanning the membrane twice with their C and N termini internally. The α-subunits have voltage-sensors in the fourth transmembrane segment and have a classical K⁺ selectivity filter. The reason for the high conductance is two rings each with 8 negative charges located at the inner and outer mouth of the pore as well as a large negatively charged outer pore vestibule accumulating the K⁺ ions (Carvacho, I. et al, Gen Physiol, 2008, 131(2), 147-161).

BK channels are unique amongst ion channels in that they are activated by depolarizing membrane potentials as well as by an increase in the intracellular Ca²⁺ concentration binding to a C-terminal site, i.e. they are voltage sensitive and calcium sensitive. This dual regulation allows BK to couple intracellular signalling to membrane potential and significantly modulate physiological responses, such as neuronal signalling and muscle contraction. In addition to this composite regulation pattern, the activity of BK channels can be further modulated by phosphorylation (protein kinases, A, C, G and CaMKII), pH, endogenous messengers (NO, cAMP, cGMP) and drugs. Since the BK channel activity is modulated by these pathways and especially by the intracellular Ca²⁺ concentration as well as by the presence of the β1 subunit, drugs interacting with these mechanisms will indirectly change the BK channel activity.

Many different chemical entities have been found to increase the activity of BK channels. Within these entities, differences in calcium dependency, subunit composition and drug binding sites have been found. Based on their origin and structure the chemical entities can be classified in: (A) endogenous BK channel modulators and structural analogues; (B) naturally occurring BK channel openers and structural analogues; (C) synthetic BK channel openers (see Nardi and Olesen, Current Medicinal Chemistry, 2008, 15, 1126-1146).

As used herein the term “BKCa channel activator” refers to any moiety, including a chemical compound, biological molecule or complex, that is capable of causing, either directly or indirectly, an increase in activity at the BKCa channel relative to baseline activity (i.e. activity in the absence of said moiety). Suitable methods for determining the activity of channels such as the BKCa channel will be familiar to a person skilled in the art. For example, the ability of a particular compound to act as a BKCa channel activator can be determined by a patch clamp experiment (see Examples section for further details). For a purported BKCa channel activator, a statistically significant increase in the number of single channel openings (spikes in the patch clamp trace) is indicative of BKCa channel activity.

Examples of BKCa channel activators suitable for use in the present invention are described in the art (see Nardi and Olesen, Current Medicinal Chemistry, 2008, 15, 1126-1146).

In on preferred embodiment, the BKCa channel activator for use according to the invention is selected from the following: TA1702 (Tanabe Seiyaku),

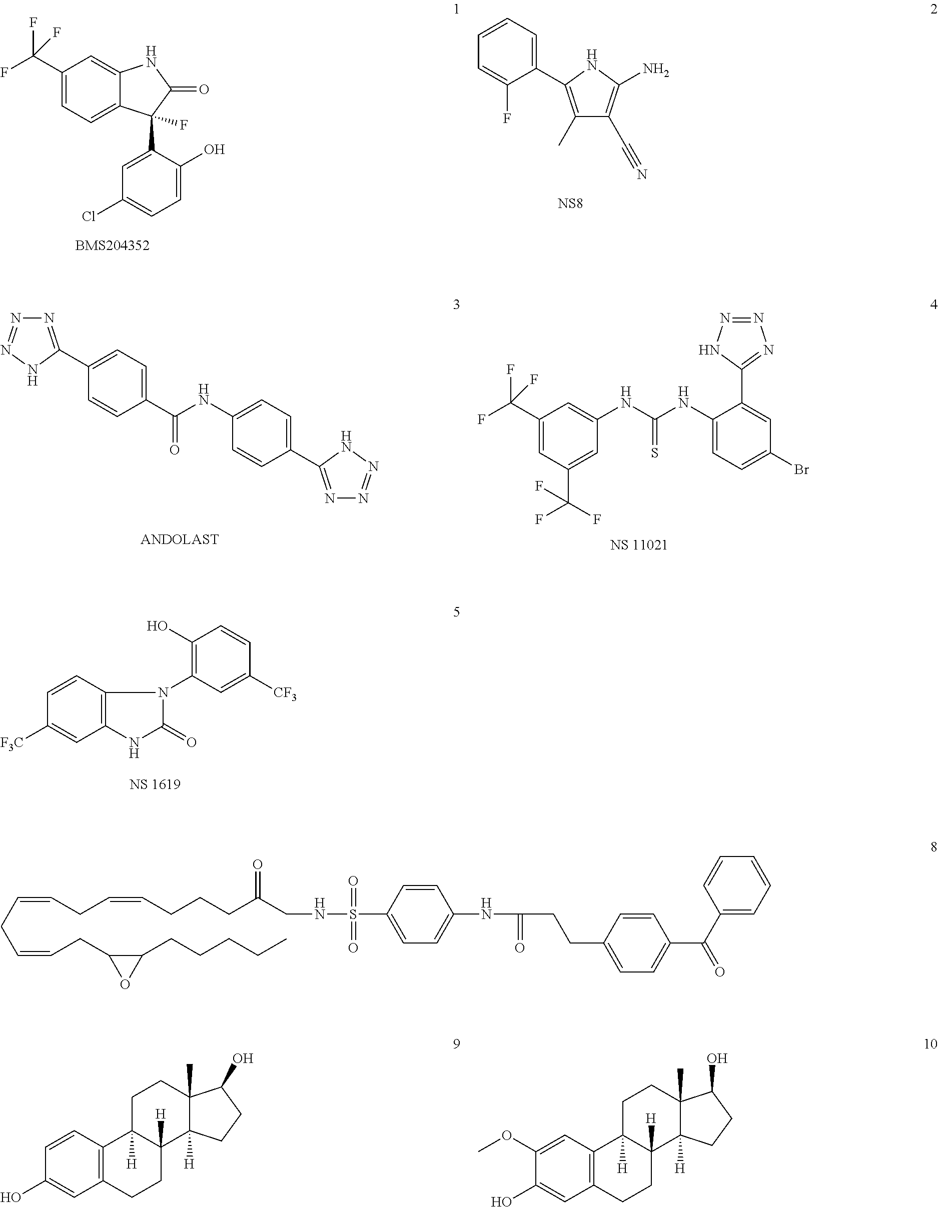

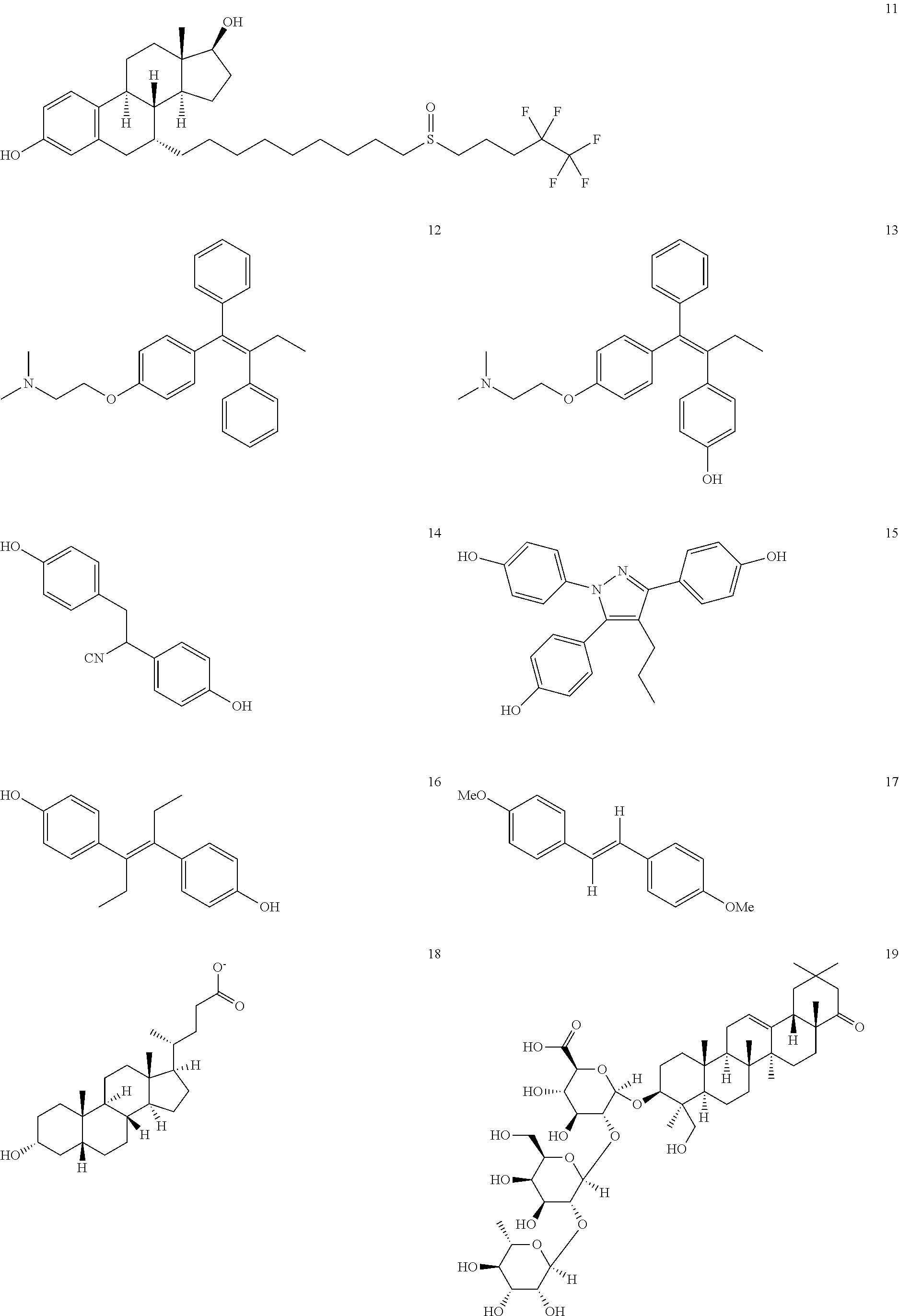

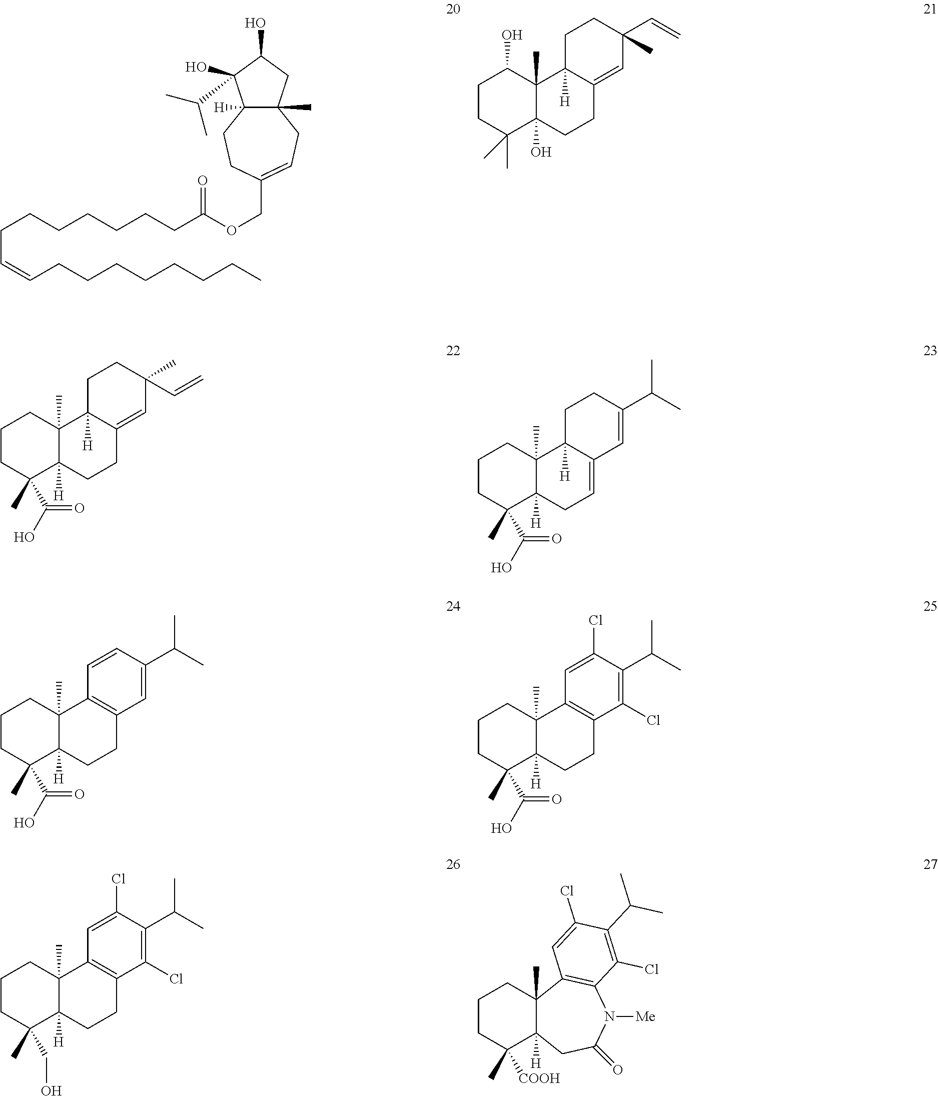

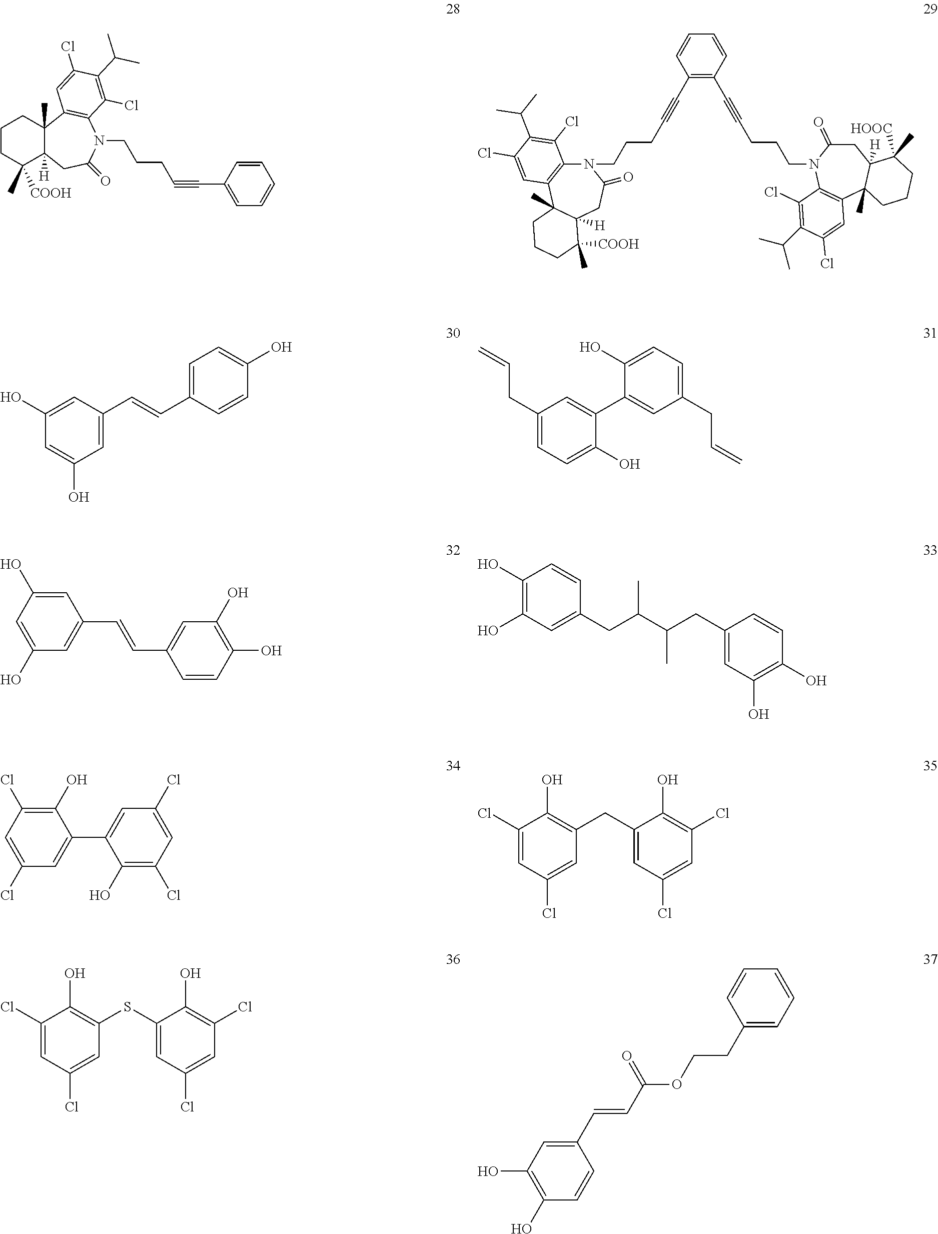

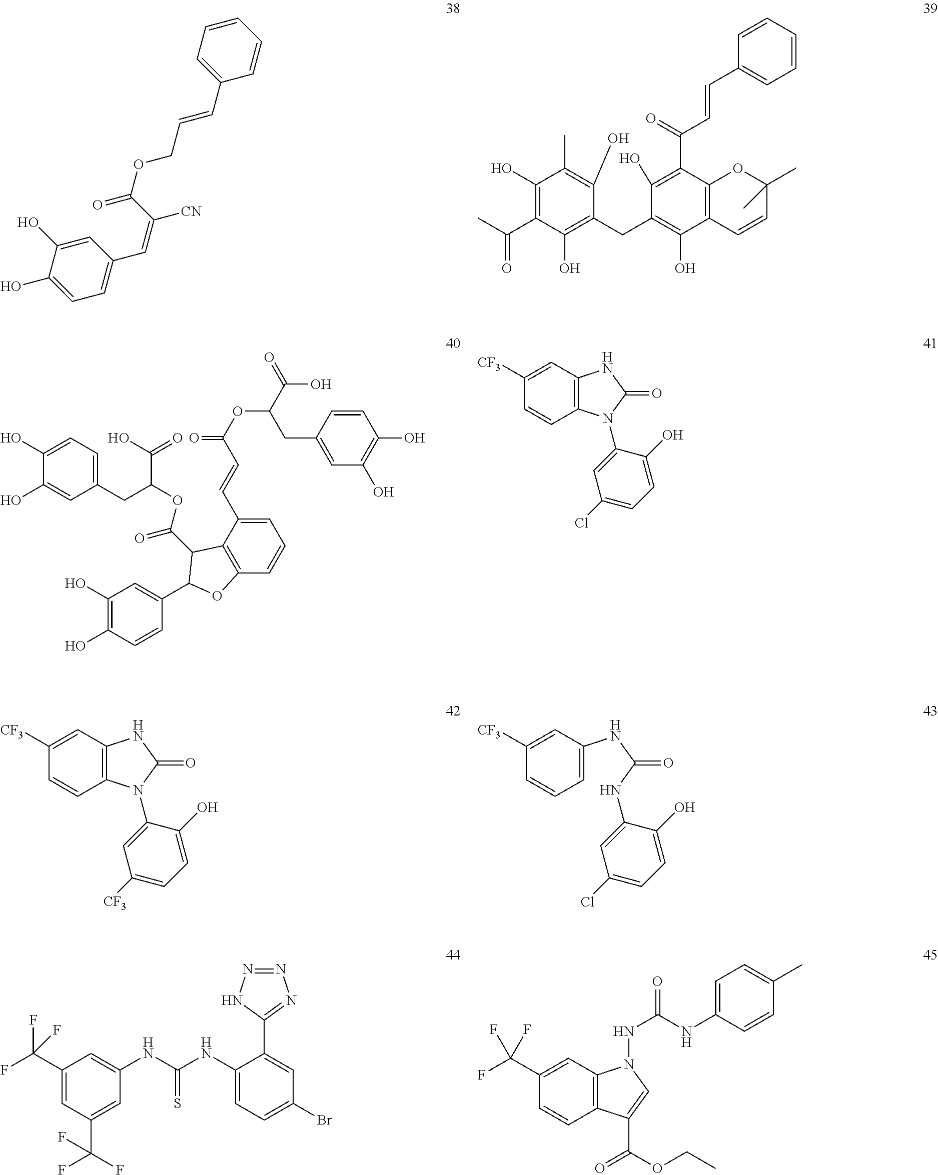

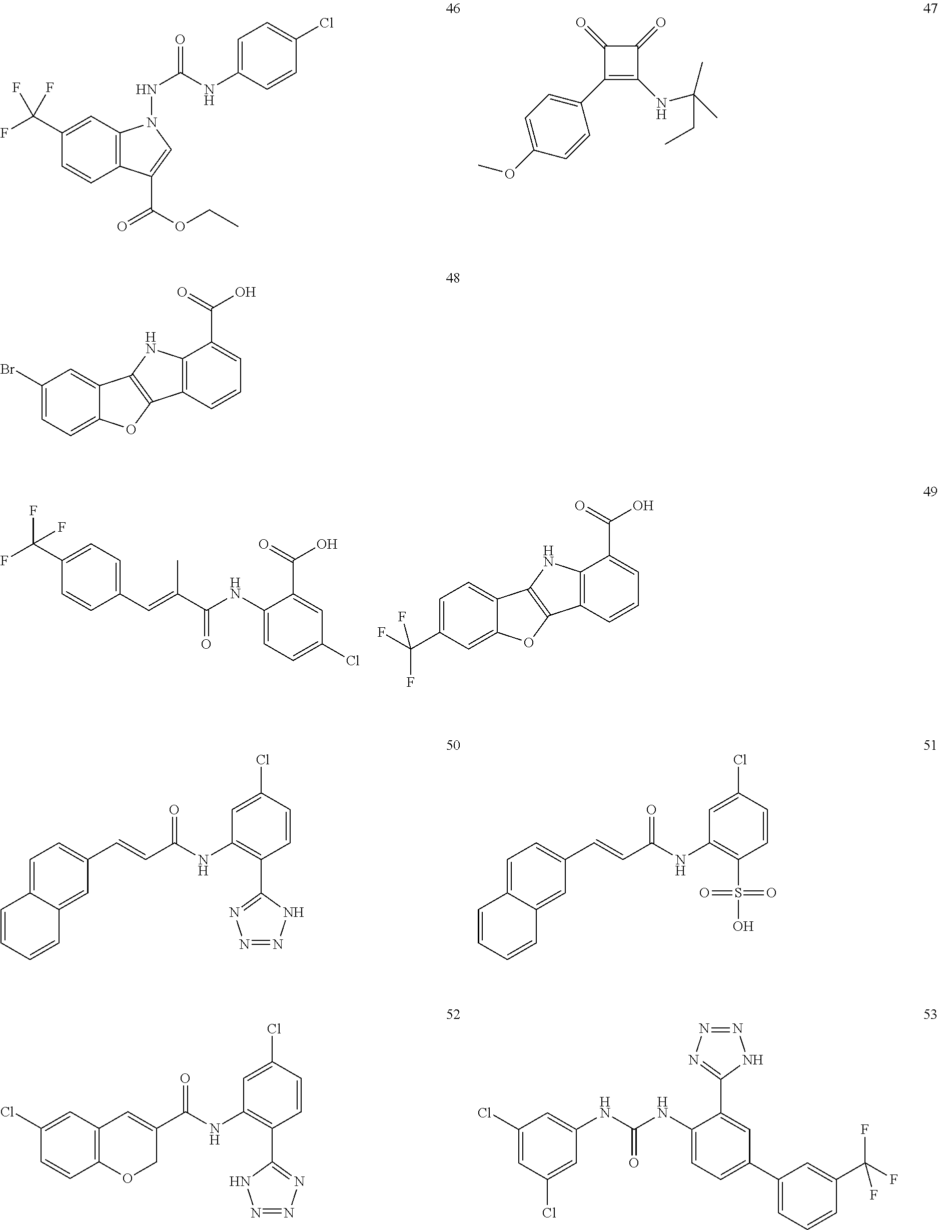

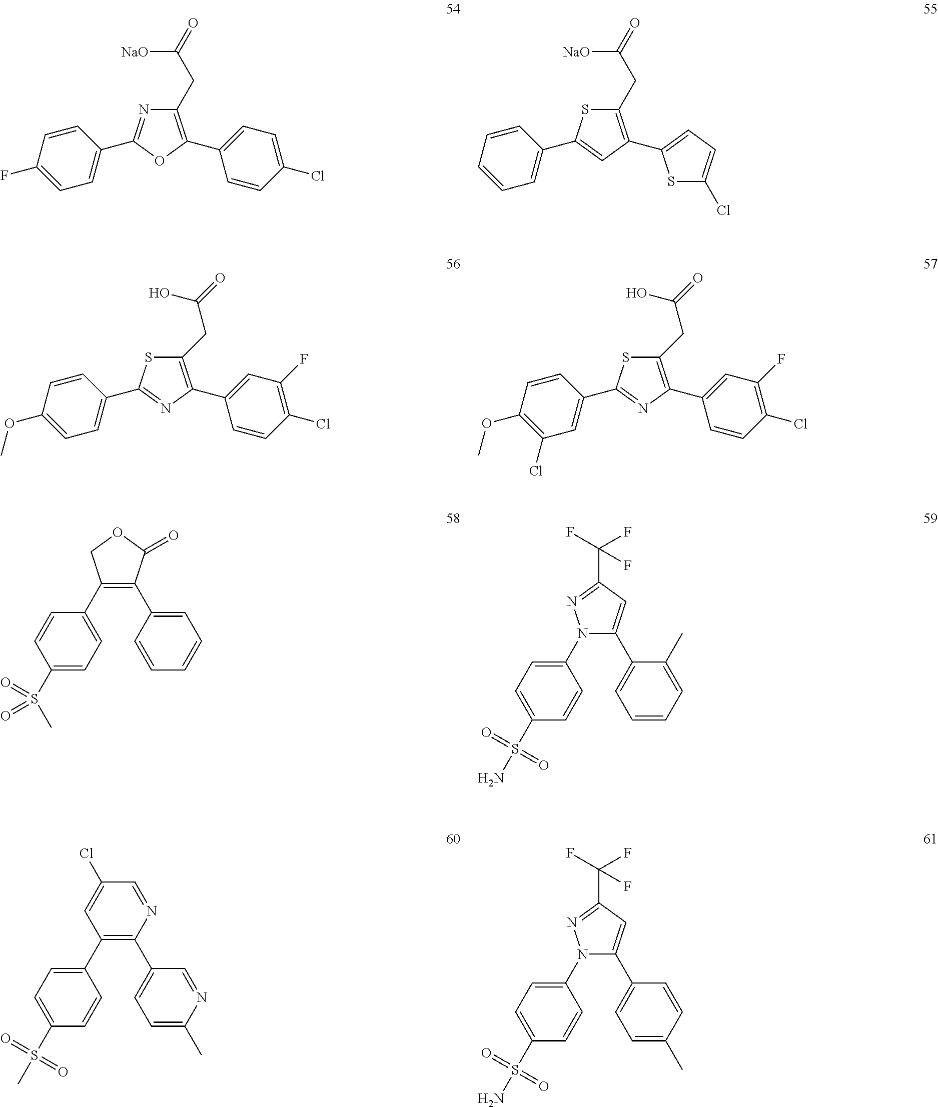

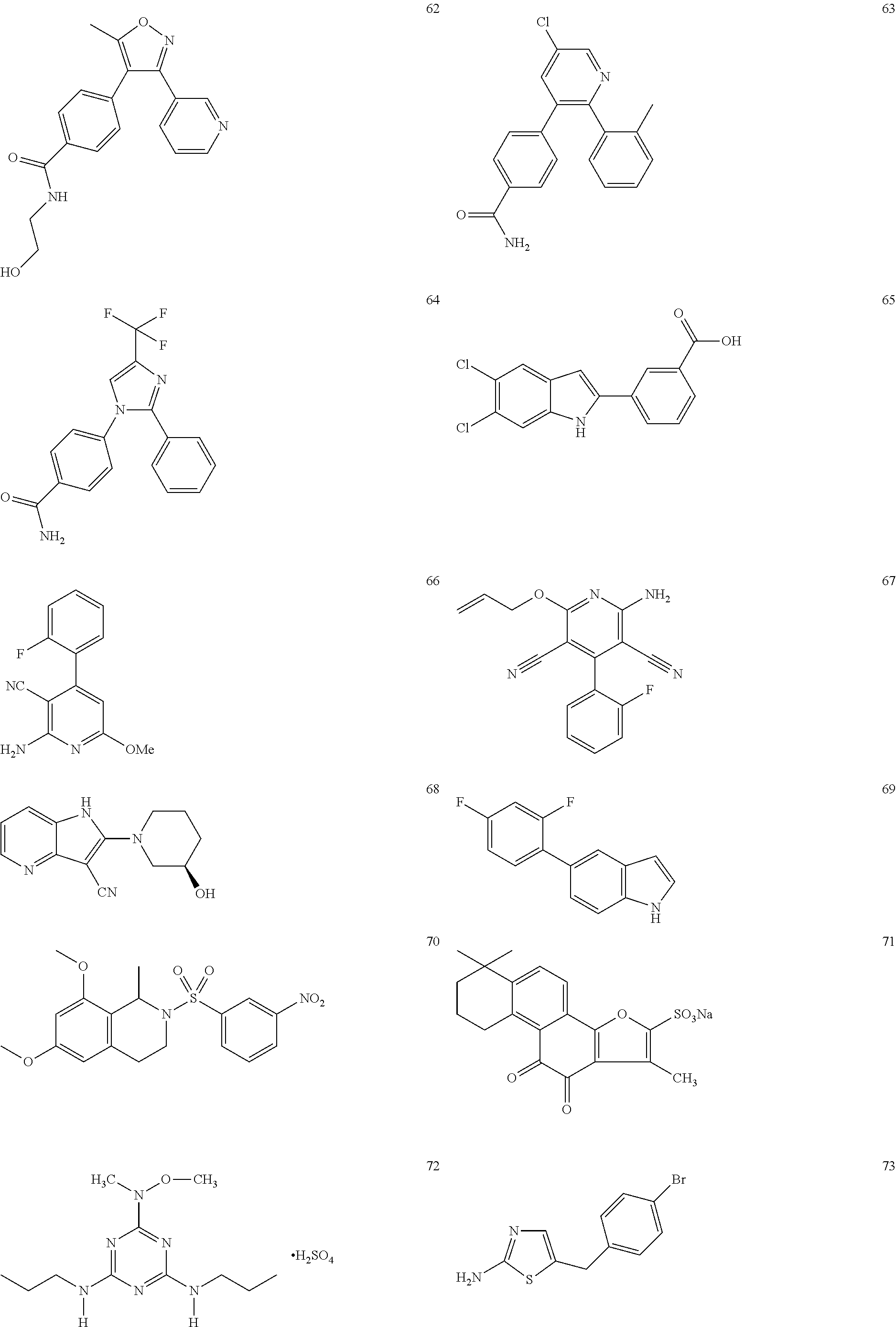

and LDD175 (4-chloro-7-trifluoromethyl-10Hbenzo[4,5]furo[3,2-b]indole-1-carboxylic acid); and pharmaceutically acceptable salts, esters or hydrates thereof.

In a preferred aspect, the invention relates to a compound selected from compounds 1-73 as recited above for use in treating a muscular disorder, and/or for use in controlling spasticity and tremors.

In one highly preferred embodiment, the BKCa channel activator for use according to the invention is selected from: TA1702 (Tanabe Seiyaku)

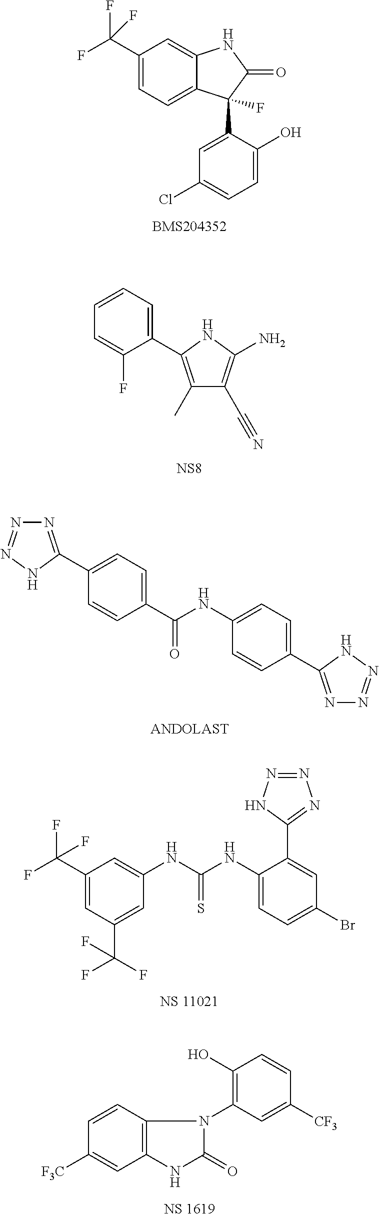

BMS 204352 (Maxipost) is the compound (3S)-3-(5-chloro-2-methoxyphenyl)-3-fluoro-1,3-dihydro-6-(trifluoromethyl)-2H-Indol-2-one (Tocris, Bristol UK).

NS 1619 is the compound 1,3-dihydro-1-[2-hydroxy-5-(trifluoromethyl)phenyl]-5-(trifluoromethyl)-2H-benzimidazol-2-one (Sigma Aldrich).

NS 11021 is the compound N′-[3,5-bis(trifluoromethyl)phenyl]-N-[4-bromo-2-(2H-tetrazol-5-yl-phenyl]thiourea (Tocris, Bristol UK).

Further details on the above compounds may be found in Nardi and Olesen (Current Medicinal Chemistry, 2008, 15, 1126-1146).

LDD175 is the compound 4-chloro-7-trifluoromethyl-10Hbenzo[4,5]furo[3,2-b]indole-1-carboxylic acid (Sung H. H., Choo S. H., Han D. H., Chae M. R., Kang S. J., Park C. S., So I., Park J. K., Lee S. W., J Sex Med. Nov. 10, 2014 doi: 10.1111/jsm.12744).

Compound 71 is Tanshinone II-A sodium sulfonate (DS-201), a water-soluble derivative of Tanshinone II-A (Tan X. Q, Cheng X. L., Yang Y., Yan L., Gu J. L., Li H., Zeng X. R., Cao J. M., Acta Pharmacol Sin. 2014 November; 35(11): 1351-63).

Compound 72 is 2-N,O-dimethylhydroxylamino-4,6-bispropylamino-5-triazine, otherwise known as GAL-021. The drug product is prepared as a H₂SO₄ salt (McLeod J F, Leempoels J M, Peng S X, Dax S L, Myers L J, Golder F J., Br J Anaesth. 2014 November; 113(5): 875-883).

In one particularly preferred embodiment, the BKCa channel activator for use according to the invention is selected from BMS 204352, NS 1619 and NS 11021.

In one highly preferred embodiment, the BKCa channel activator for use according to the invention is BMS 204352.

In another highly preferred embodiment, the BKCa channel activator for use according to the invention is NS 1619.

In another highly preferred embodiment, the BKCa channel activator for use according to the invention is NS 11021.

### Therapeutic Applications

The present invention relates to BKCa activators for use in treating a muscular disorder, for use in controlling spasticity and tremors.

One preferred embodiment relates to a BKCa activator for use in treating a muscular disorder

The term “muscular disorder” is used in a broad sense to cover any muscular disorder or disease, in particular a neurological disorder or disease, more particularly, a neurodegenerative disease or an adverse condition involving neuromuscular control. Thus, the term includes, for example, multiple sclerosis (MS), spasticity, Parkinson's disease, Huntingdon's Chorea, spinal cord injury, including spinal cord spasticity, and Tourettes' syndrome.

In one preferred embodiment, the muscular disorder is a disorder of skeletal muscle. Skeletal muscle is a form of striated muscle tissue which is under the control of the somatic nervous system; that is to say, it is voluntarily controlled.

Preferably, the muscular disorder is a neuromuscular disorder.

Another preferred embodiment relates to a BKCa activator for use in treating a tremor.

Another preferred embodiment relates to a BKCa activator for use in controlling spasticity.

In one highly preferred embodiment, the BKCa activator is for use in treating spasticity in MS.

Spasticity in MS is characterised by stiffness in one or more muscle groups, due to over excitation. It may be accompanied by spasms, which are often painful, and controlled movement becomes difficult. Spasticity is a common feature of MS with 40-84% of patients reporting mild to severe spasticity in different studies (Barnes M P, Kent R M, Semlyen J K, McMullen K M (2003), Neurorehabil Neural Repair 17:66-70; Hemmett L, Holmes J, Barnes M, Russell N (2004), QJM 97:671-676; Rizzo M A, Hadjimichael O C, Preiningerova J, Vollmer T L (2004), Mult Scler 10:589-595; Collongues N, Vermersch P (2013), Expert Rev Neurother 13:21-25, 2013; Oreja-Guevara C, Gonzalez-Segura D, Vila C (2013), Int J Neurosci 123:400-408). Spasticity in MS is associated with a decrease in patient life quality. Current drugs used to treat spasticity include Baclofen, a GABA_{B} agonist, Tizanidine, an alpha2 adrenergic agonist, Dantrolene, a drug that acts on muscle sarcolamella and nabiximols, a cannabinoid receptor 1 (CB1) agonist. All these drugs show less than optimal control of symptoms and are accompanied by moderate to severe side effects such as sedation, muscle weakness or have the potential for abuse. Thus poor tolerance and under-treatment result in unmet needs in MS spasticity management.

In one highly preferred embodiment, the BKCa activator is for use in treating spinal cord spasticity. As used herein, “spinal cord spasticity” refers to skeletal muscle overactivity that occurs when communication between the brain and spinal cord is disrupted by a spinal cord injury, other injury or illness.

Another aspect relates to the use of a BKCa channel activator in the preparation of a medicament for treating a muscular disorder, or for treating or controlling spasticity or tremors.

As used herein the phrase “preparation of a medicament” includes the use of a BK activator directly as the medicament in addition to its use in a screening programme for further agents or in any stage of the manufacture of such a medicament.

Another aspect of the invention relates to a method of treating a muscular disorder, or for treating or controlling spasticity or tremors in a subject in need thereof, said method comprising administering to the subject a therapeutically effective amount of a BKCa channel activator.

### Pharmaceutical Compositions

Even though the BKCa activators described herein (including their pharmaceutically acceptable salts, esters and pharmaceutically acceptable solvates) can be administered alone, they will generally be administered in admixture with a pharmaceutical carrier, excipient or diluent, particularly for human therapy. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol and sorbitol. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate and sodium chloride.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

### Salts/Esters

The BKCa activators described herein can be present as salts or esters, in particular pharmaceutically acceptable salts or esters.

Pharmaceutically acceptable salts of the BKCa activators of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. hydrohalic acids (such as hydrochloride, hydrobromide and hydrolodide), sulphuric acid, phosphoric acid sulphate, bisulphate, hemisulphate, thiocyanate, persulphate and sulphonic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with amino acids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

### Enantiomers/Tautomers

In all aspects of the present invention previously discussed, the invention includes, where appropriate all enantiomers and tautomers of the BKCa activators described herein. The man skilled in the art will recognise compounds that possess optical properties (one or more chiral carbon atoms) or tautomeric characteristics. The corresponding enantiomers and/or tautomers may be isolated/prepared by methods known in the art. Thus, the invention encompasses the enantiomers and/or tautomers in their isolated form, or mixtures thereof, such as for example, racemic mixtures of enantiomers.

### Stereo and Geometric Isomers

Some of the BKCa activators of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those inhibitor agents, and mixtures thereof. The terms used in the claims encompass these forms, provided said forms retain the appropriate functional activity (though not necessarily to the same degree).

The present invention also includes all suitable isotopic variations of the BKCa activators described herein, or pharmaceutically acceptable salts thereof. An isotopic variation of a BKCa activator of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

### Solvates

The present invention also includes solvate forms of the BKCa activators described herein. The terms used in the claims encompass these forms.

Polymorphs

The invention furthermore relates to the BKCa activators described herein in their various crystalline forms, polymorphic forms and (an)hydrous forms. It is well established within the pharmaceutical industry that chemical compounds may be isolated in any of such forms by slightly varying the method of purification and or is isolation form the solvents used in the synthetic preparation of such compounds.

### Prodrugs

The invention further includes the the BKCa activators described herein in prodrug form. Such prodrugs are generally compounds wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above, for example, methyl or ethyl esters of the acids), wherein the reversion may be carried out be an esterase etc. Other such systems will be well known to those skilled in the art.

In one highly preferred embodiment, the prodrug is an ester of said BK activator, more preferably a methyl or ethyl ester.

### Administration

The pharmaceutical compositions of the present invention may be adapted for oral, rectal, vaginal, parenteral, intramuscular, intraperitoneal, intraarterial, intrathecal, intrabronchial, subcutaneous, topical, intradermal, intravenous, nasal, buccal or sublingual routes of administration. The skilled person will be familiar with preferred formulation types for many of the BKCa channel activators described herein.

For oral administration, particular use is made of compressed tablets, pills, tablets, gellules, drops, and capsules. Preferably, these compositions contain from 1 to 250 mg and more preferably from 10-100 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose. In addition, the compositions may be formulated as extended release formulations.

### Dosage

A person of ordinary skill in the art can easily determine an appropriate dose of one of the instant compositions to administer to a subject without undue experimentation. The skilled person will be familiar with preferred formulation types for many of the BKCa channel activators described herein. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from about 0.01 to about 30 mg/kg body weight, such as from about 0.1 to about 10 mg/kg, more preferably from about 0.1 to about 1 mg/kg body weight. In one highly preferred embodiment, the dose is from about 2 to about 6 mg/kg body weight, more preferably, about 5 mg/kg body weight.

In an exemplary embodiment, one or more doses of 10 to 150 mg/day will be administered to the patient.

### Combinations

In a particularly preferred embodiment, the one or more BKCa activators described herein are administered in combination with one or more other pharmaceutically active agents. In such cases, the compounds of the invention may be administered consecutively, simultaneously or sequentially with the one or more other pharmaceutically active agents.

For example, in one preferred embodiment, the BKCa activator is administered in combination with Baclofen. Baclofen, also known as Chlorophenibut (brand names Kemstro, Lioresal, Liofen, Gablofen, Lyflex, Beklo and Baclosan) is a derivative of gamma-aminobutyric acid (GABA) that is used to treat spasticity. It is a GABA receptor agonist that is understood to exert beneficial effects by virtue of its action at spinal and supraspinal sites.

In another preferred embodiment, the BKCa activator is administered in combination with tizanidine. Tizanidine is a centrally acting α2 adrenergic agonist that is used as a muscle relaxant.

The present invention is further described by way of example, and with reference to the following figures wherein:

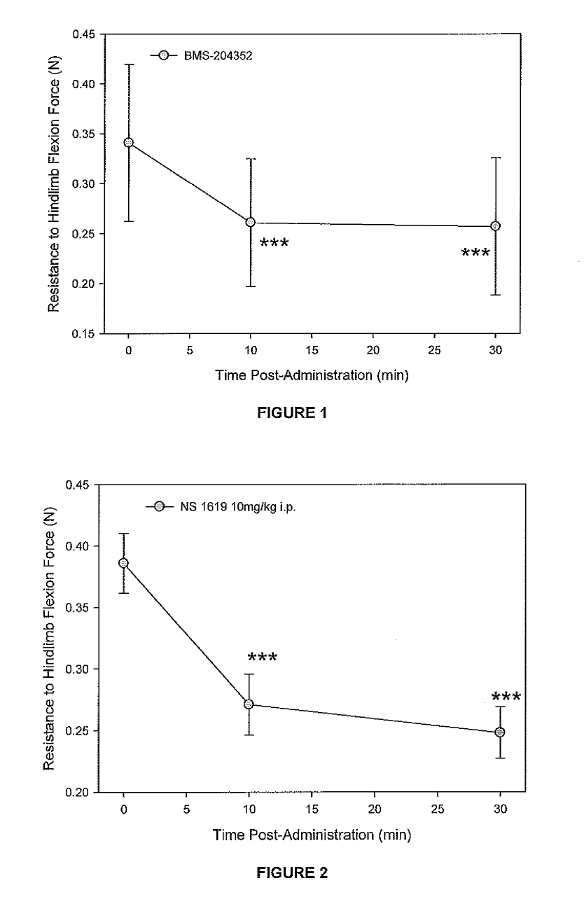

FIG. 1 shows the resistance to hindlimb flexion force (N) against time post-administration (minutes) for mice injected i.p. with 20 mg/kg BMS 204352 (n=11).

FIG. 2 shows the resistance to hindlimb flexion force (N) against time post-administration (minutes) for mice injected i.p. with 10 mg/kg NS 1619.

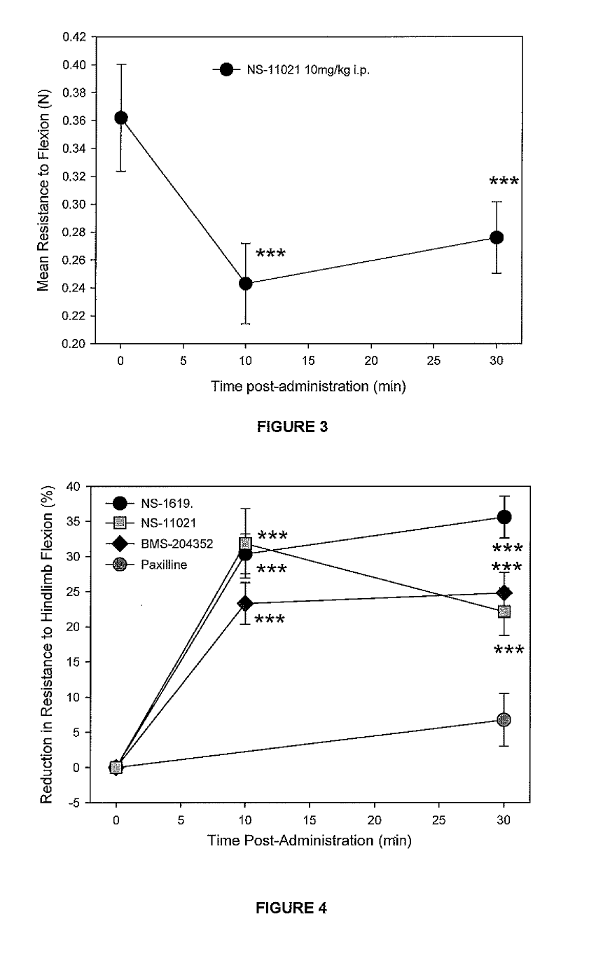

FIG. 3 shows the mean resistance to flexion (N) against time post-administration (minutes) for mice injected i.p. with 10 mg/kg NS 11021.

FIG. 4 shows the reduction in resistance to hindlimb flexion (%) against time post-administration (minutes) for mice injected i.p. with NS 1619, NS 11021, BMS 204352 or Paxilline.

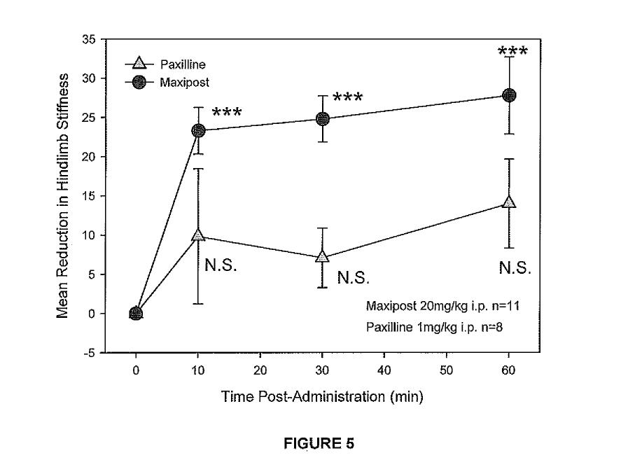

FIG. 5 shows the mean reduction in hindlimb stiffness (%) against time post-administration (minutes) for mice injected i.p. with 20 mg/kg Maxipost (BMS 204352) or 1 mg/kg Paxilline.

### EXAMPLES

### Chemicals

BMS 204352, (3S)-3-(5-Chloro-2-methoxyphenyl)-3-fluoro-1,3-dihydro-6-(trifluoromethyl)-2H-Indol-2-one, was purchased from Tocris, Bristol UK.

NS 1619, 1,3-dihydro-1-[2-hydroxy-5-(trifluoromethyl)phenyl]-5-(trifluoromethyl)-2H-benzimidazol-2-one, was purchased from Sigma Aldrich.

BMS 191011, 3-[(5-chloro-2-hydroxyphenyl)methyl]-5-[4-(trifluoromethyl)phenyl]-1,3,4-oxadiazol-2(3H)-one, was purchased from Tocris, Bristol UK or Sigma Aldrich.

Paxilline, (2R,4bS,6aS,12bS,12cR,14aS)-5,6,6a,7,12,12b,12c,13,14,14a-Decahydro-4b-hydroxy-2-(1-hydroxy-1-methylethyl)-12b,12c-dimethyl-2H pyrano[2″,3″:5″,6′]benz [1′,2′:6,7]indeno[1,2-b]indol-3(4bH)-one (a BK antagonist) was purchased from Tocris, Bristol UK or Sigma Aldrich.

BMS 204352 and NS 1619 were dissolved in ethanol:cremophor:phosphate buffered saline 1:1:18. These were injected intraperitoneally in amounts based on previously published data: 10 mg/kg i.p. of NS 1619 (CNS excluded; Lu R. et al, Pain, 2014; 155: 556-565) and 20 mg/kg i.p. of BMS 204352 (CNS penetrant, but well tolerated; Korsgaard M. P. et al, J Pharmacol Exp Ther. 2005; 314: 282-92; Gribkoff V. K. et al, Nat Med. 2001; 7: 471-477; Kristensen L. V. et al, Neurosci Lett. 2011; 488: 178-82; Krishna R. et al, Biopharm Drug Dispos. 2002; 23: 227-231) were used as proof of concept. Paxilline was used at 1 mg/kg, just below doses reported to cause tremors in mice.

### Spasticity is Inhibited by BK Channel Openers

Biozzi ABH were injected with mouse spinal cord homogenate in Freunds adjuvant on day 0 and day 7 based on published protocols (Al-Izki S. et al. Mult Scler Rel Dis. 2012; 1: 29-38 and relapsing progressive experimental autoimmune encephalomyelitis developed. Following the development of visible spasticity animals were randomly allocated to treatment groups and spasticity was assessed by measuring limb stiffness against a strain guage (Pryce G. et al. FASEB J. 28: 2014; 117-130).

Animals were injected with:
BMS 204352: 20 mg/kg i.p. n=11 (FIG. 1; Table 2);
NS 1619: 10 mg/kg i.p. n=13 (FIG. 2; Table 1);
NS 11021: 10 mg/kg i.p. n=13 (FIG. 3; Table 3)
   in ethanol:cremophor:PBS (1:1:18).

Limb stiffness was assessed using a strain gauge before and following treatment. **P<0.001 compared to baseline (0 min) using repeated measures ANOVA.

FIG. 4 shows the reduction in resistance to hindlimb flexion (%) versus time post administration (minutes) for animals injected i.p. with NS-1619 (10 mg/kg i.p. n=13; Table 1), NS-11021 (10 mg/kg i.p. n=13; Table 3), BMS 204352 (20 mg/kg i.p. n=11; Table 2) or paxilline (a BK antagonist; 1 mg/kg i.p. n=14).

FIG. 5 shows the mean reduction in hindlimb stiffness (%) versus time post administration (minutes) for animals injected with BMS 204352 (20 mg/kg i.p. n=11) or paxilline (1 mg/kg i.p. n=8).

### Patch Clamp Analysis

### Cell Culture

The human umbilical vein derived endothelial cell line, EA.hy926 (Edgell et al., 1983) at passage >45 was grown in DMEM containing 10% FCS and 1% HAT (5 mM hypoxanthine, 20 μM aminopterin, 0.8 mM thymidine) and cells were maintained in an incubator at 37° C. in 5% CO₂ atmosphere. Cells were plated on either 10 mm (for patch-clamp recordings) or 30 mm glass cover slips (for Ca²⁺ measurements).

### Electrophysiological Recordings

Membrane potential of EA.hy926 cells was recorded using nystatin-perforated patch clamp technique as described previously (Bondarenko et al, 2010, Br. J. Pharmacol. 161, 308-320). For membrane potential recordings from EA.hy926 cells the standard bath solution contained (in mM): 140 NaCl, 5 KCl, 1.2 MgCl₂, 10 HEPES, 10 glucose, 2.4 CaCl₂, patch pipettes were filled with a solution containing (in mmol/L): 140 KCl; 0.2 EGTA; 10 HEPES (pH adjusted to 7.2 using KOH). The resistance of the pipettes was 3-5 MΩ for whole cell and 6-8 MΩ for single channel recordings.

Single-channel recordings were obtained from excised inside-out membrane patches in symmetrical solutions. The pipettes were filled with (in mM) 140 KCl, 10 HEPES, 1 MgCl₂, 5 EGTA, 4,931 CaCl₂ with pH 7.2 by adding KOH (i.e. 10 μM free Ca²⁺, G. Droogmans, Leuven, Belgium; ftp://ftp.cc.kuleuven.ac.be/pub/droogmans/cabuf.zip). Cells were perfused with a standard bath solution containing (in mM) 140 NaCl, 5 KCl, 1.2 MgCl₂, 10 HEPES, 10 glucose, 2.4 CaCl₂. Following gigaseal formation, bath solution was switched to the following (in mM) 140 KCl, 10 HEPES, 1 MgCl₂, 5 EGTA and a desired free Ca²⁺ concentration which was adjusted by adding different amounts of CaCl₂ calculated by the program CaBuf. pH was adjusted to 7.2 by adding KOH. Membrane currents and potential were recorded using a List EPC7 amplifier (List, Germany) and pClamp (version 8.2, Axon Instruments) software. The identity of the channel is confirmed by its conductance characteristics and voltage and calcium sensitivity, and also to its sensitivity to inhibitors including paxilline, iberotoxin, martinotoxin and Charybdotoxin. For purported BKCa channel activators, a statistically significant increase in the number of single channel openings (spikes in the patch clamp trace) is indicative of BKCa channel activity.

Various modifications and variations of the described methods of the invention will be apparent to those skilled in the art without departing from the scope and spirit of the invention. Although the invention has been described in connection with specific preferred embodiments, various modifications of the described modes for carrying out the invention which are obvious to those skilled in chemistry or related fields are intended to be within the scope of the following claims.

TABLE 1
Resistance to flexion forces (N) for animals injected with NS 1619
(10 mg/kg i.p. n = 13 limbs) in ethanol:cremophor:PBS (1:1:18)
(limb stiffness was assessed using a strain gauge of individual
left and right legs before and following treatment.
Resistance to Flexion Forces (N)

| Limb | 0 min | 10 min | 30 min |
|---|---|---|---|
| 0l | 0.2539 | 0.1992 | 0.2061 |
| 0r | 0.2669 | 0.1914 | 0.2028 |
| 1l | 0.3604 | 0.2422 | 0.2315 |
| 1r | 0.5796 | 0.5005 | 0.4435 |
| 2l | 0.3956 | 0.2022 | 0.2002 |
| 2r | 0.3258 | 0.2094 | 0.2169 |
| 3l | 0.3389 | 0.1914 | 0.1582 |
| 3r | 0.4053 | 0.3341 | 0.1945 |
| 4l | 0.3985 | 0.2422 | 0.2637 |
| 4r | 0.4307 | 0.3188 | 0.2757 |
| 5l | 0.3526 | 0.2618 | 0.2188 |
| 5r | 0.4961 | 0.3671 | 0.3464 |
| 6r | 0.4149 | 0.2669 | 0.2718 |

**P < 0.001 compared to baseline (0 min) using repeated measures ANOVA).

TABLE 2
Resistance to flexion forces (N) for animals injected with BMS 204352
(20 mg/kg i.p. n = 11 limbs) in ethanol:cremophor:PBS (1:1:18)
(limb stiffness was assessed using a strain gauge of individual
left and right legs before and following treatment.
Resistance to Flexion Forces (N)

| 0 min | 10 min | 30 min | 60 min |
|---|---|---|---|
| 0.3839 | 0.3040 | 0.2738 | 0.2891 |
| 0.3708 | 0.2769 | 0.2625 | 0.2599 |
| 0.2519 | 0.1732 | 0.1859 | 0.1576 |
| 0.2765 | 0.2244 | 0.2149 | 0.2129 |
| 0.2920 | 0.2686 | 0.2276 | 0.2745 |
| 0.3057 | 0.2344 | 0.2051 | 0.2158 |
| 0.3933 | 0.3533 | 0.3151 | 0.3534 |
| 0.5033 | 0.3732 | 0.4158 | 0.3318 |
| 0.2882 | 0.1805 | 0.1735 | 0.0980 |
| 0.2680 | 0.2235 | 0.2601 | 0.2347 |
| 0.4202 | 0.2595 | 0.2876 | 0.2819 |

**P < 0.001 compared to baseline (0 min) using repeated measures ANOVA)

TABLE 3
Resistance to flexion forces (N) for animals injected with
NS 11021 (10 mg/kg i.p. n = 13 limbs n = 7 animals) in
ethanol:cremophor:PBS (1:1:18) and limb stiffness was
assessed using a strain gauge of individual left and
right legs before and following treatment.
Restistance to Flexion Force (N)

| | 0 min | 10 min | 30 min |
|---|---|---|---|
| 1l | 0.2393 | 0.1905 | 0.2285 |
| 1r | 0.3785 | 0.2296 | 0.3210 |
| 2l | 0.2520 | 0.1553 | 0.1514 |
| 2r | 0.3363 | 0.1528 | 0.2191 |
| 3l | 0.2598 | 0.2022 | 0.2315 |
| 3r | 0.4654 | 0.2867 | 0.3122 |
| 4l | 0.2891 | 0.2207 | 0.2139 |
| 4r | 0.4391 | 0.2599 | 0.3183 |
| 5l | 0.2637 | 0.2032 | 0.2178 |
| 5r | 0.6498 | 0.3644 | 0.4917 |
| 6l | 0.2549 | 0.2696 | 0.2559 |
| 6r | 0.6015 | 0.5137 | 0.4079 |
| 7r | 0.2770 | 0.1093 | 0.2156 |

**P < 0.001 compared to baseline (0 min) using repeated measures ANOVA.

What is claimed is:

1. A method of treating a muscular disorder in a subject in need thereof, comprising administering to the subject a BKCa channel activator.

2. The method according to claim 1 wherein the muscular disorder is a disorder of skeletal muscle.

3. The method according to claim 1 wherein the muscular disorder is a neuromuscular disorder.

4. A method of treating or controlling spasticity or tremors in a subject in need thereof, comprising administering to the subject a BKCa channel activator.

5. The method according to claim 4, for treating spasticity in multiple sclerosis (MS).

6. The method according to claim 4, for treating spinal cord spasticity.

7. The method according to claim 1 wherein the BKCa channel activator is selected from the following:
TA1702 (Tanabe Seiyaku) 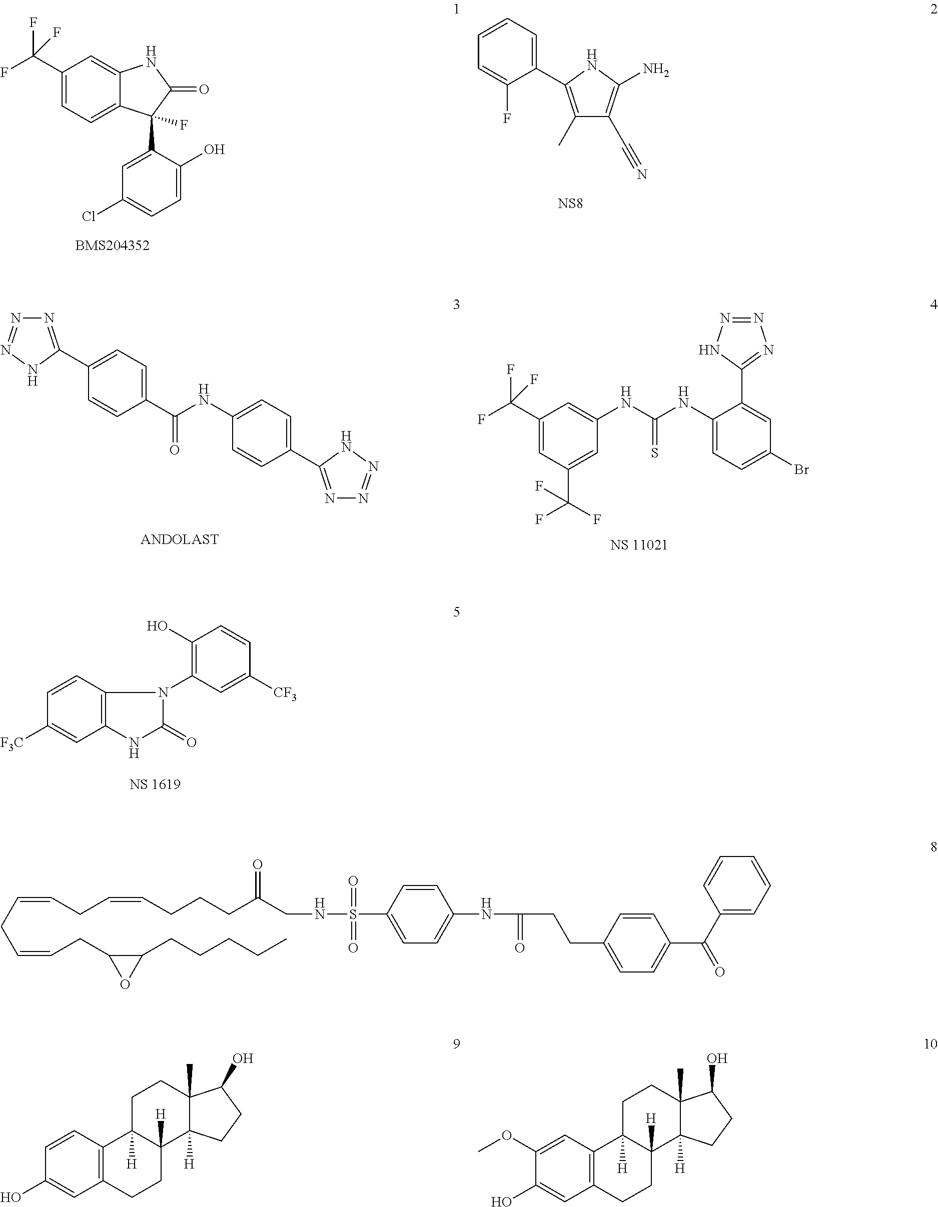 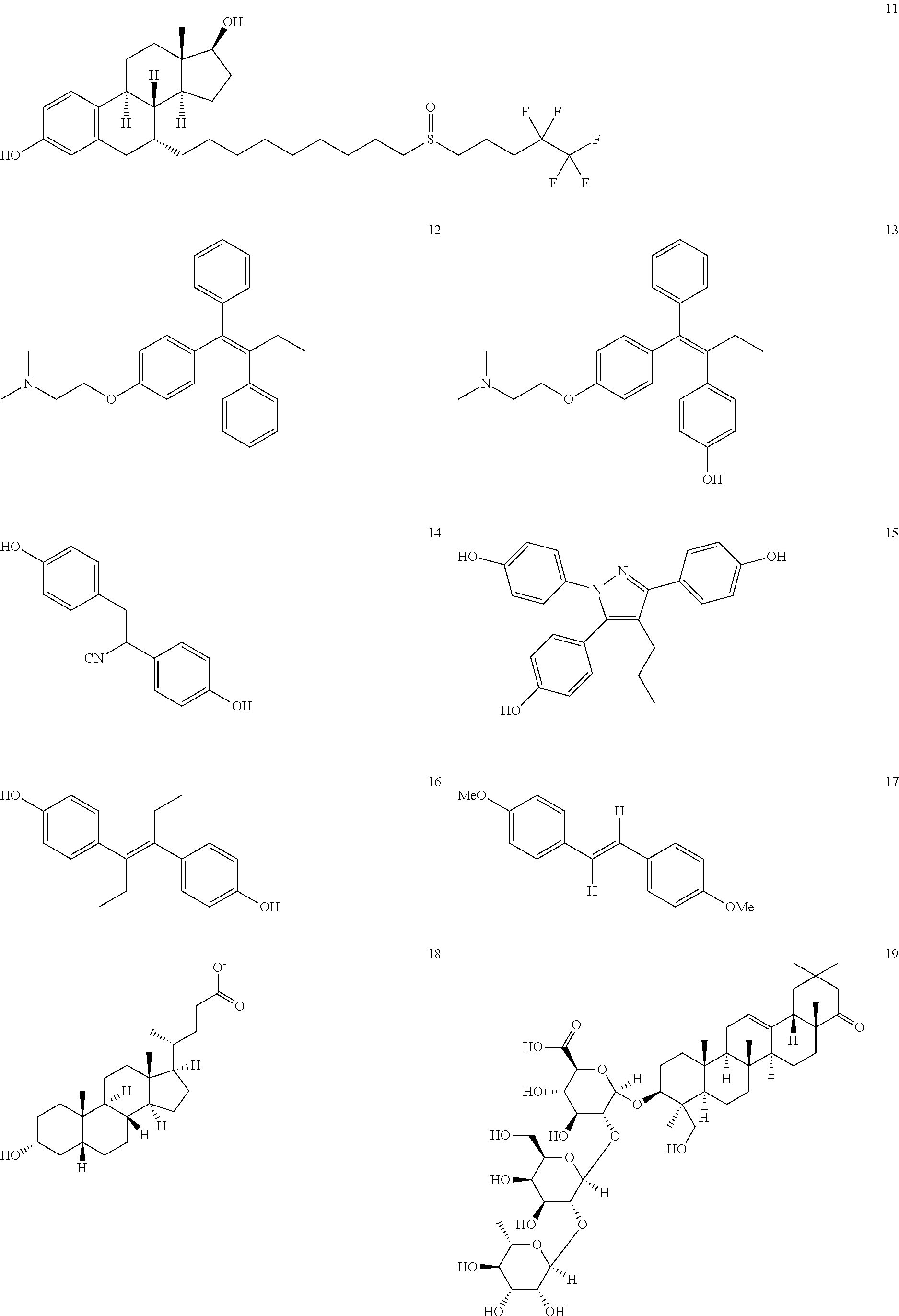 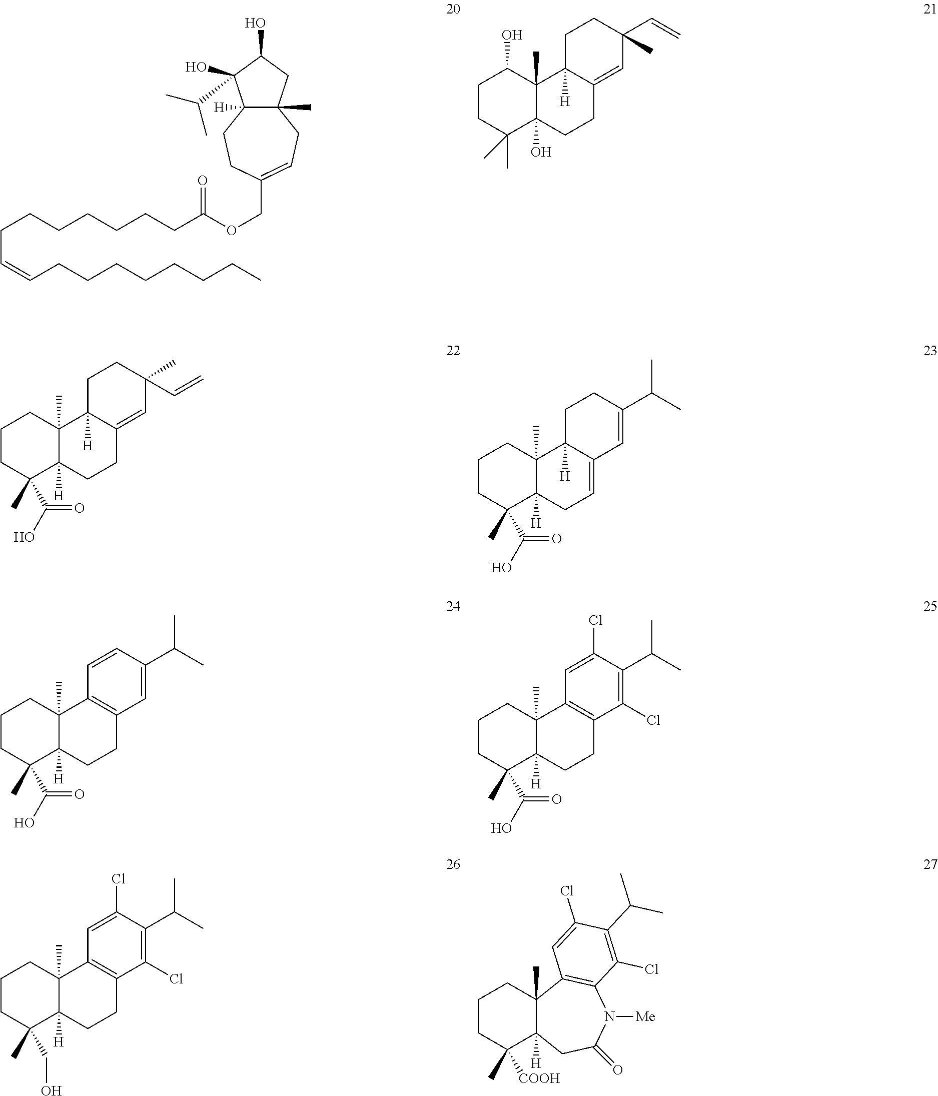 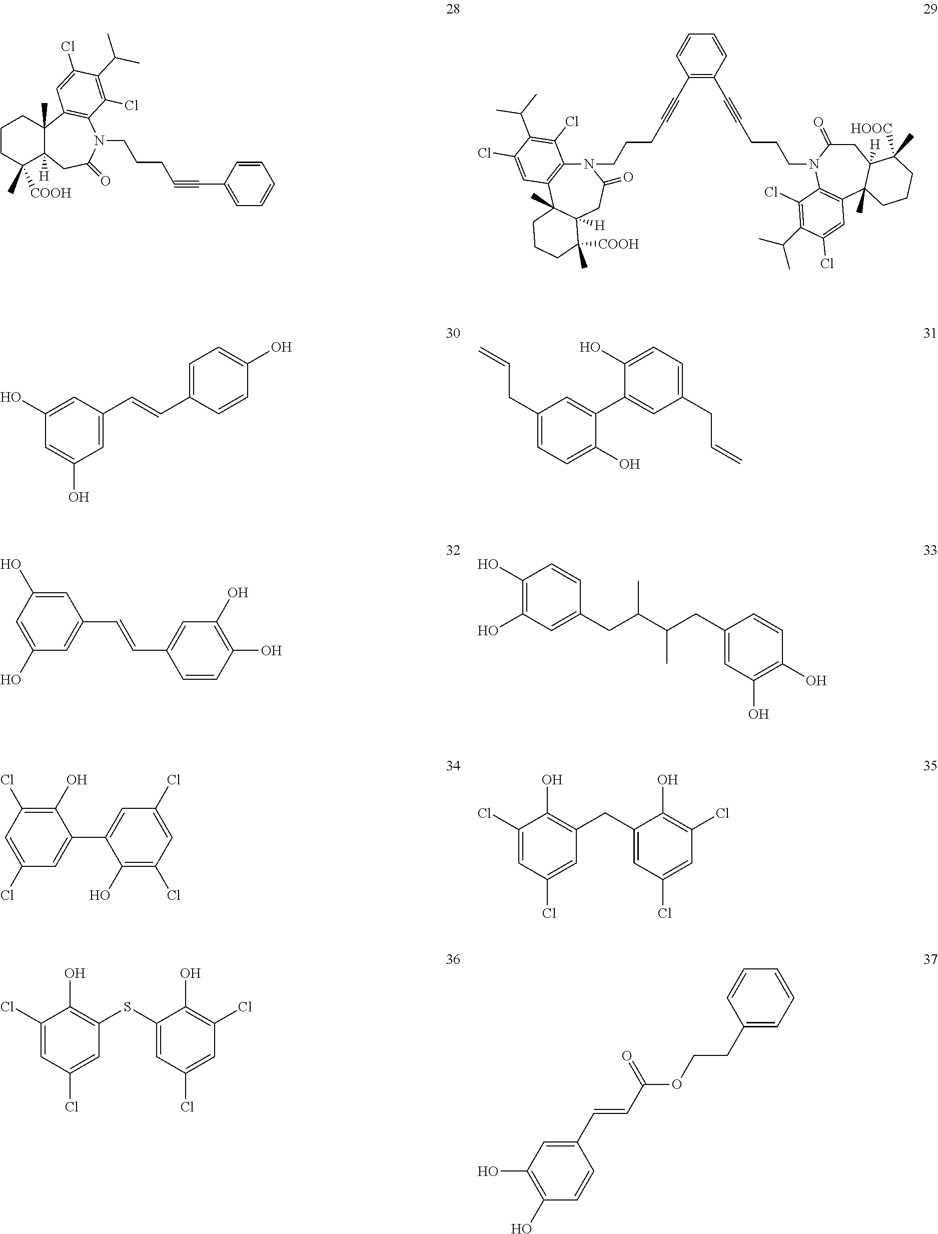 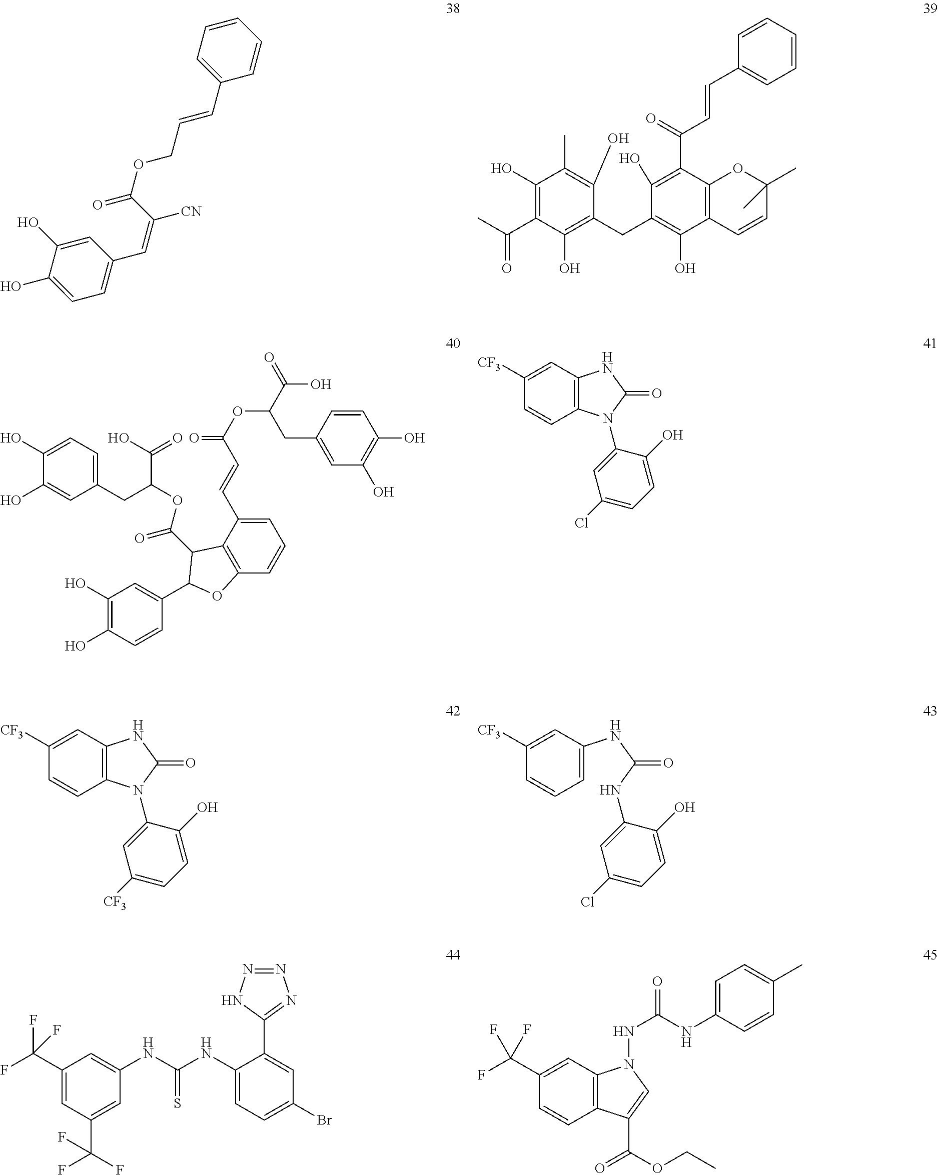 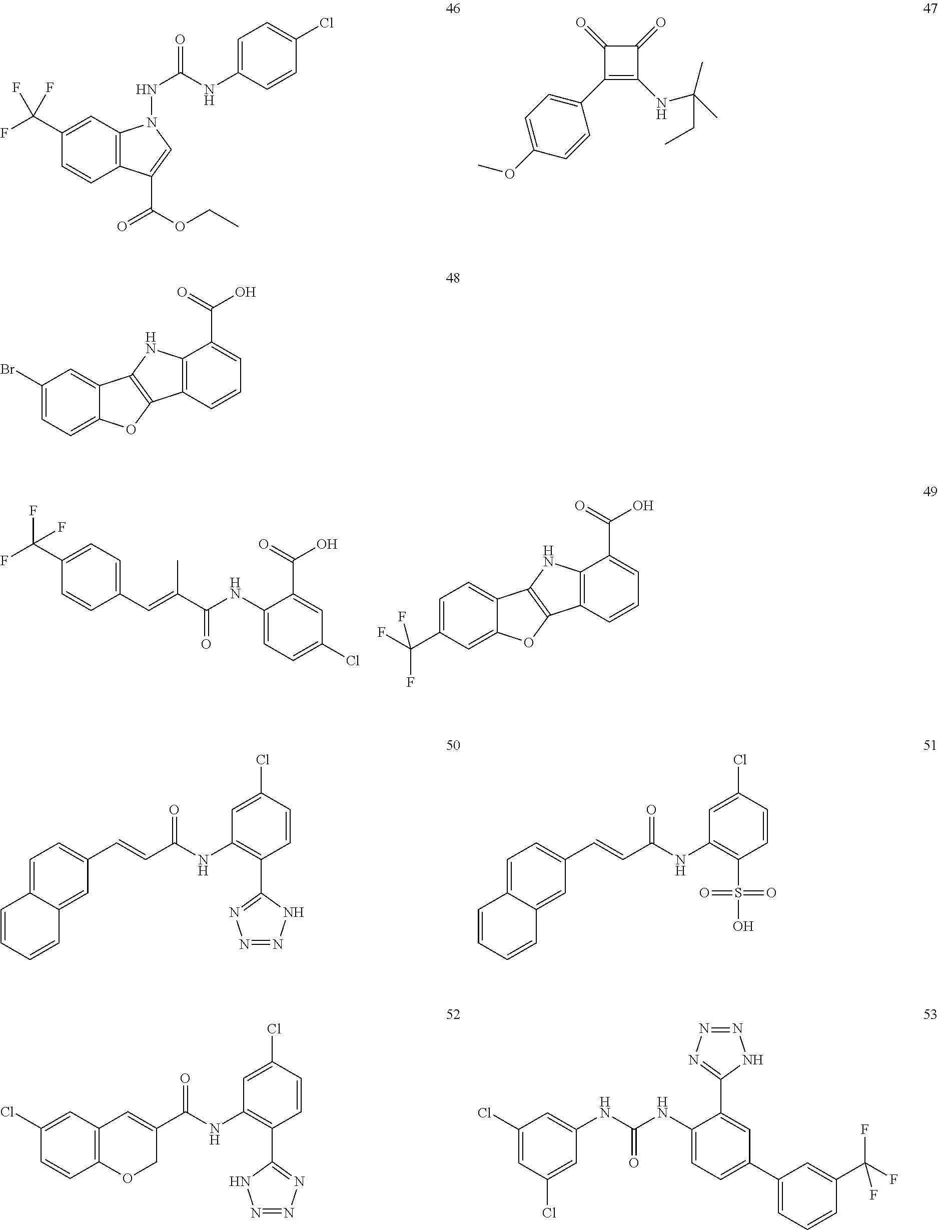 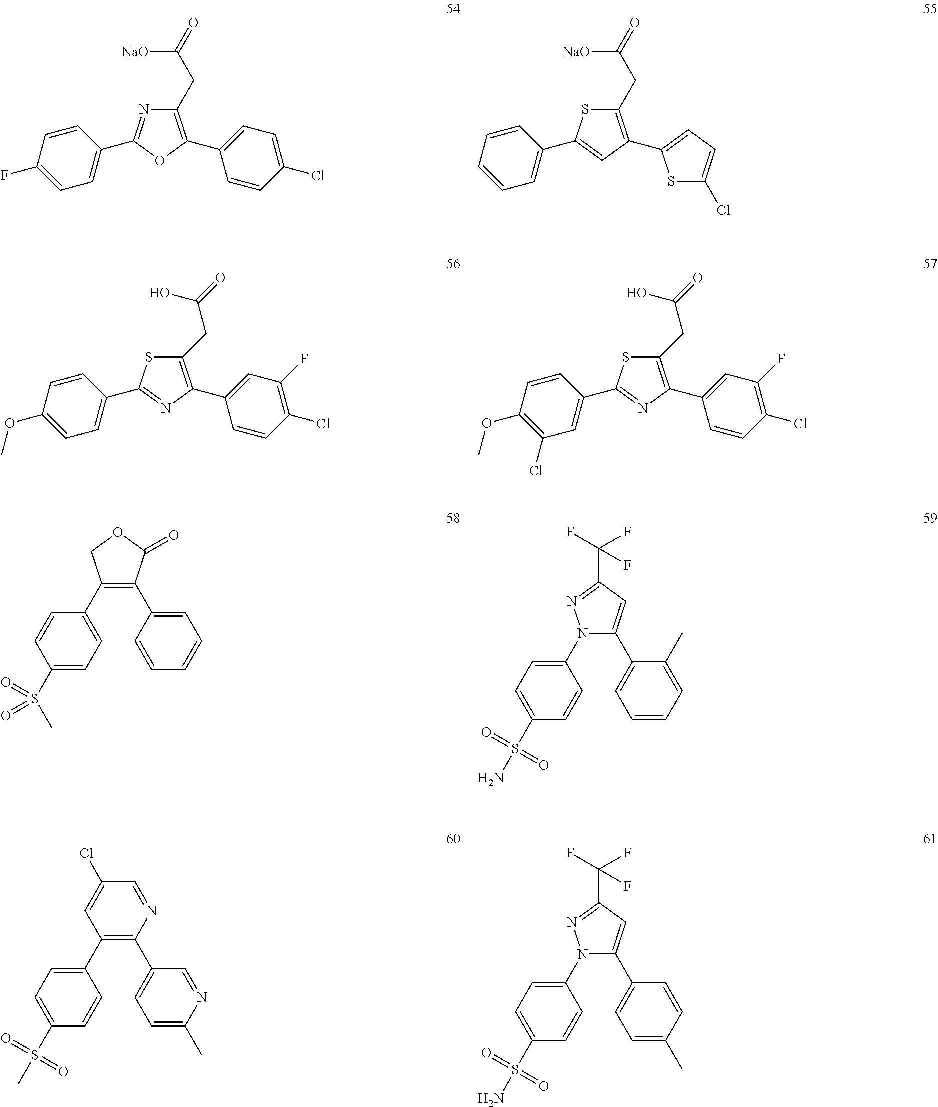 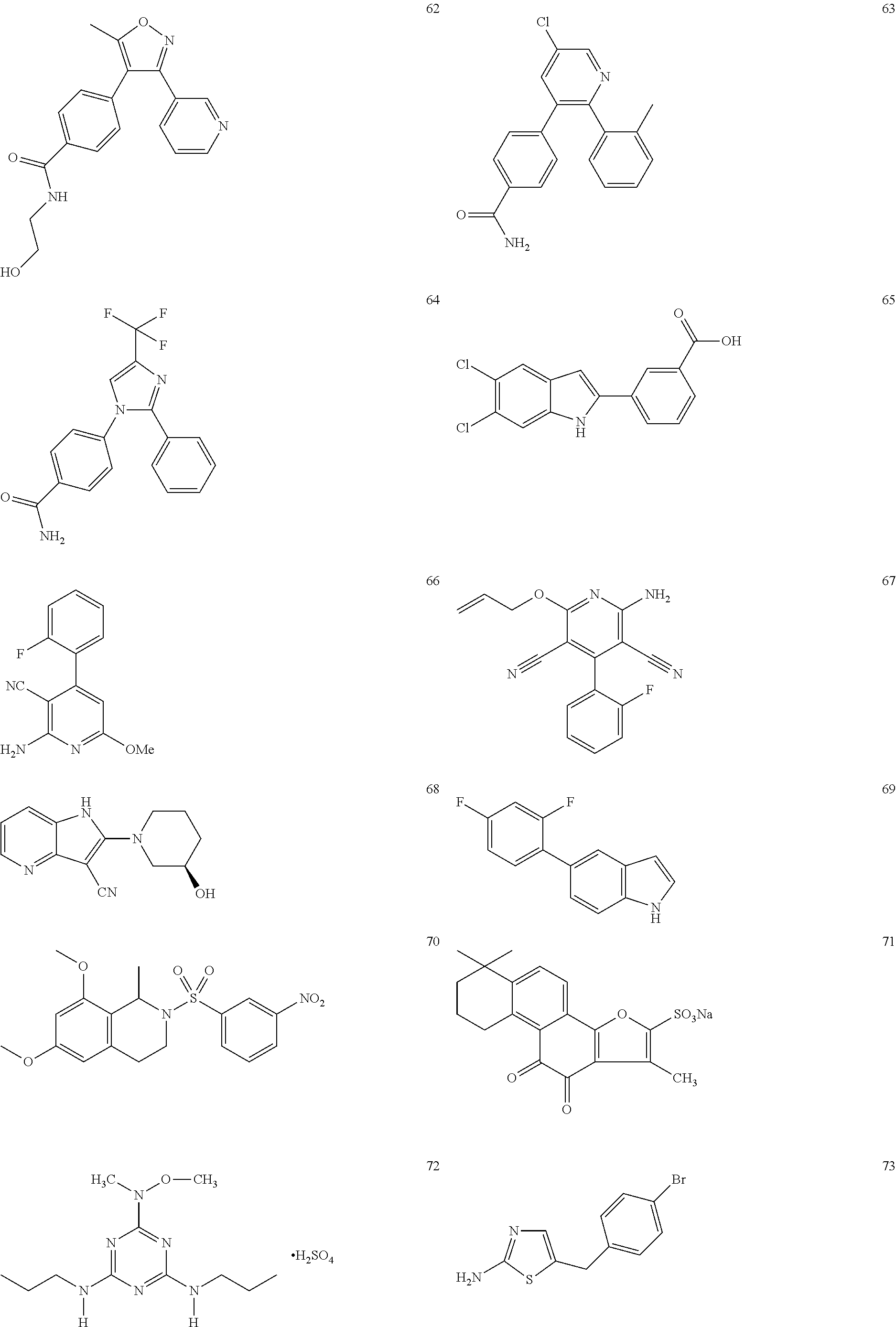
or LDD175 (4-chloro-7-trifluoromethyl-10Hbenzo[4,5]furo[3,2-b]indole-1-carboxylic acid);
or pharmaceutically acceptable salts, esters or hydrates thereof.

8. The method according to claim 1 wherein the BKCa channel activator is selected from: TA1702 (Tanabe Seiyaku);

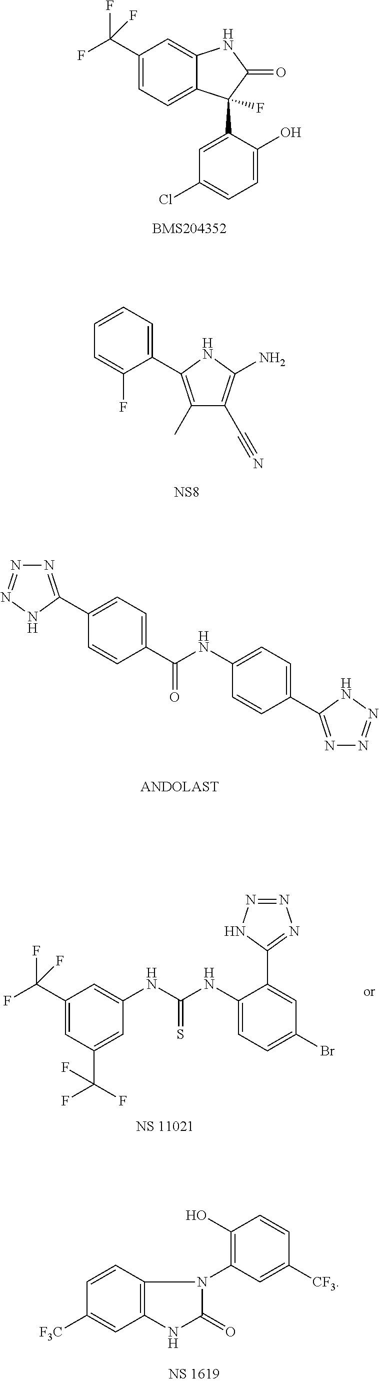

9. The method according to claim 1 wherein the BKCa channel activator is in admixture with a pharmaceutically acceptable diluent, excipient or carrier.

10-12. (canceled)

13. A method of treating or controlling spasticity or tremors in a subject in need thereof, said method comprising administering to the subject a therapeutically effective amount of a BKCa channel activator selected from the following:
TA1702 (Tanabe Seiyaku); 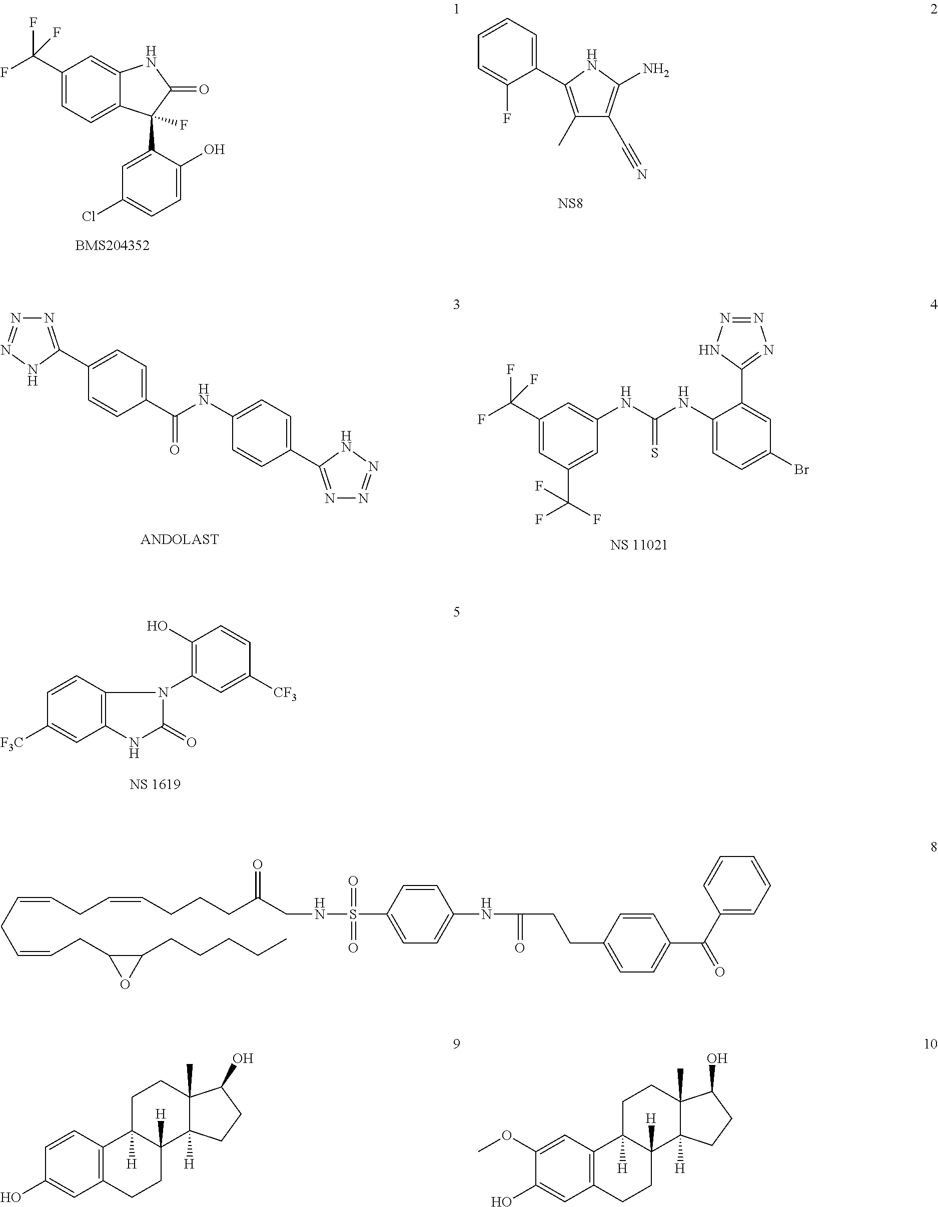 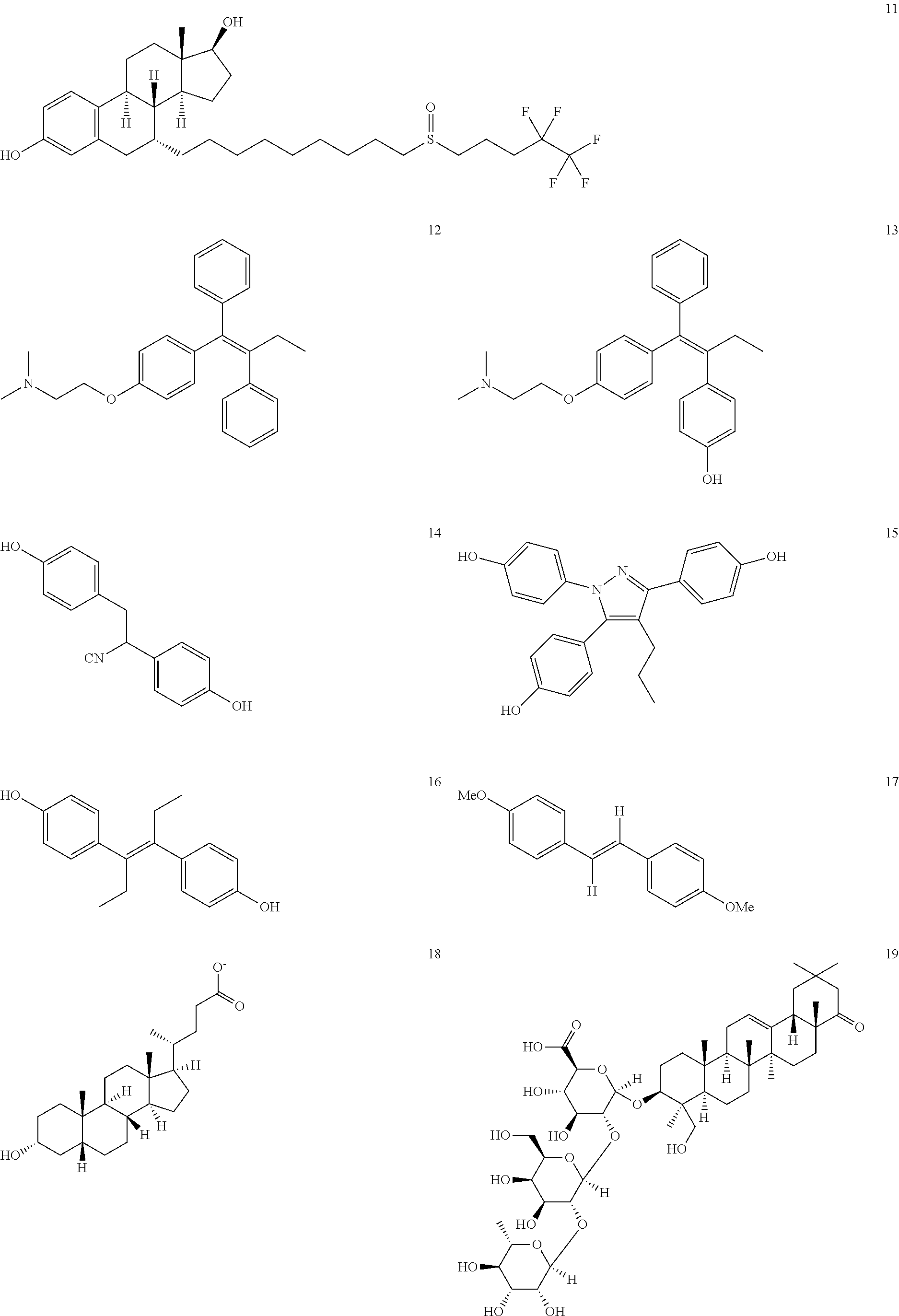 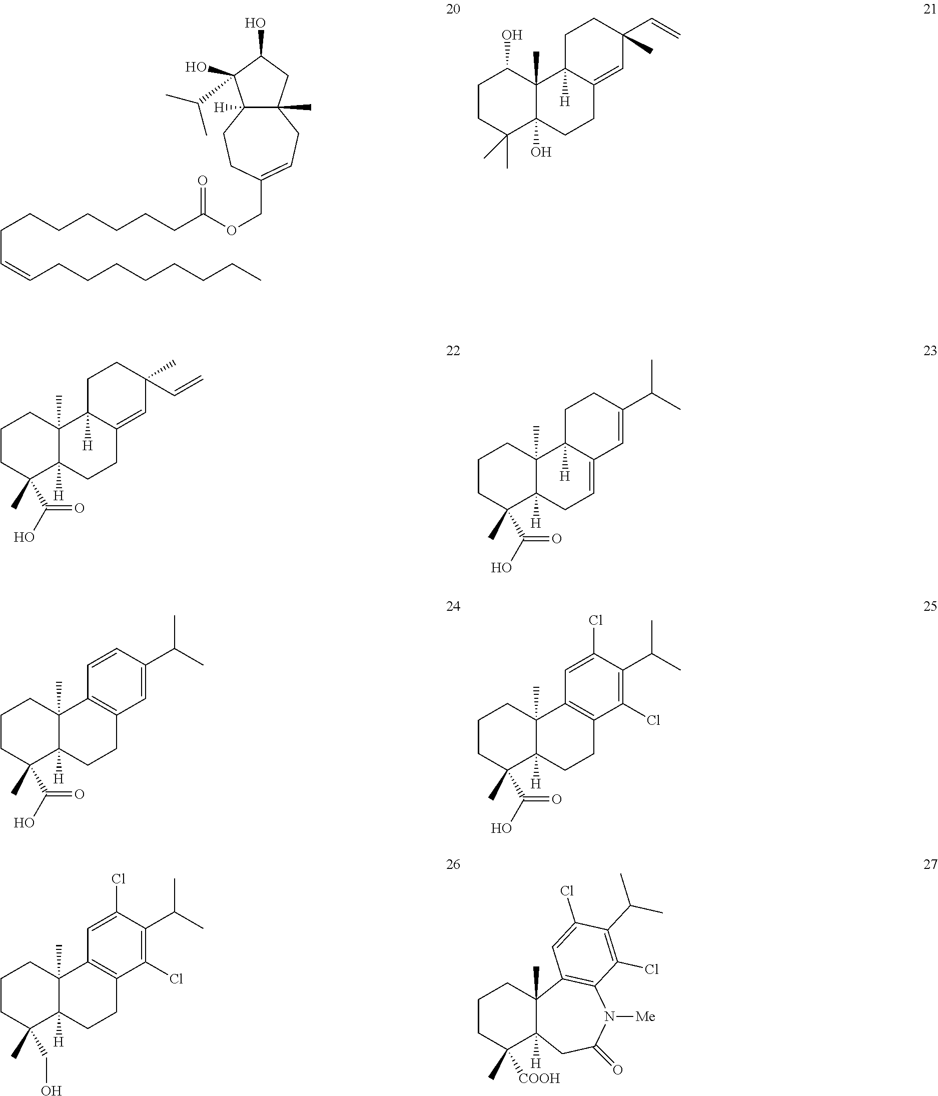 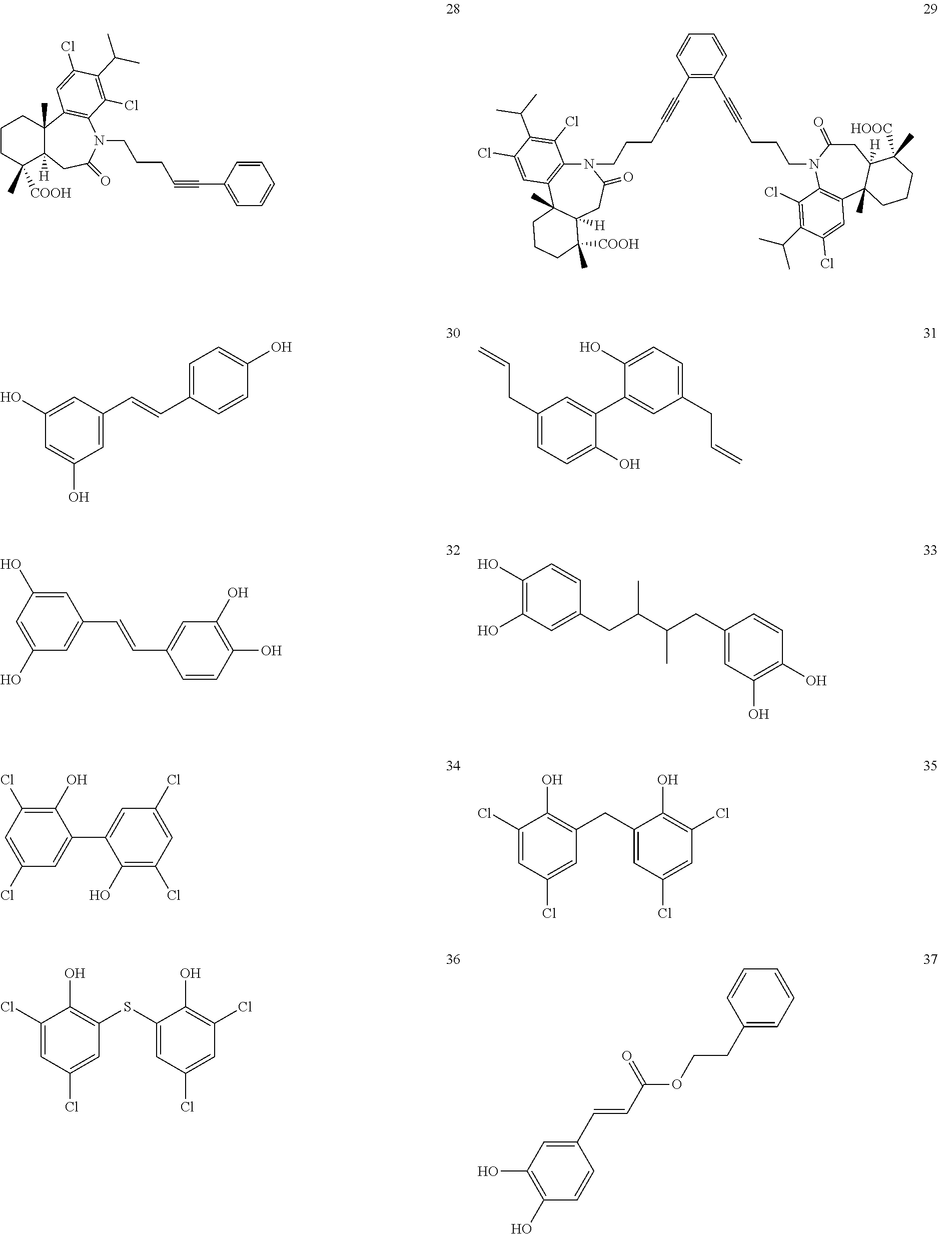 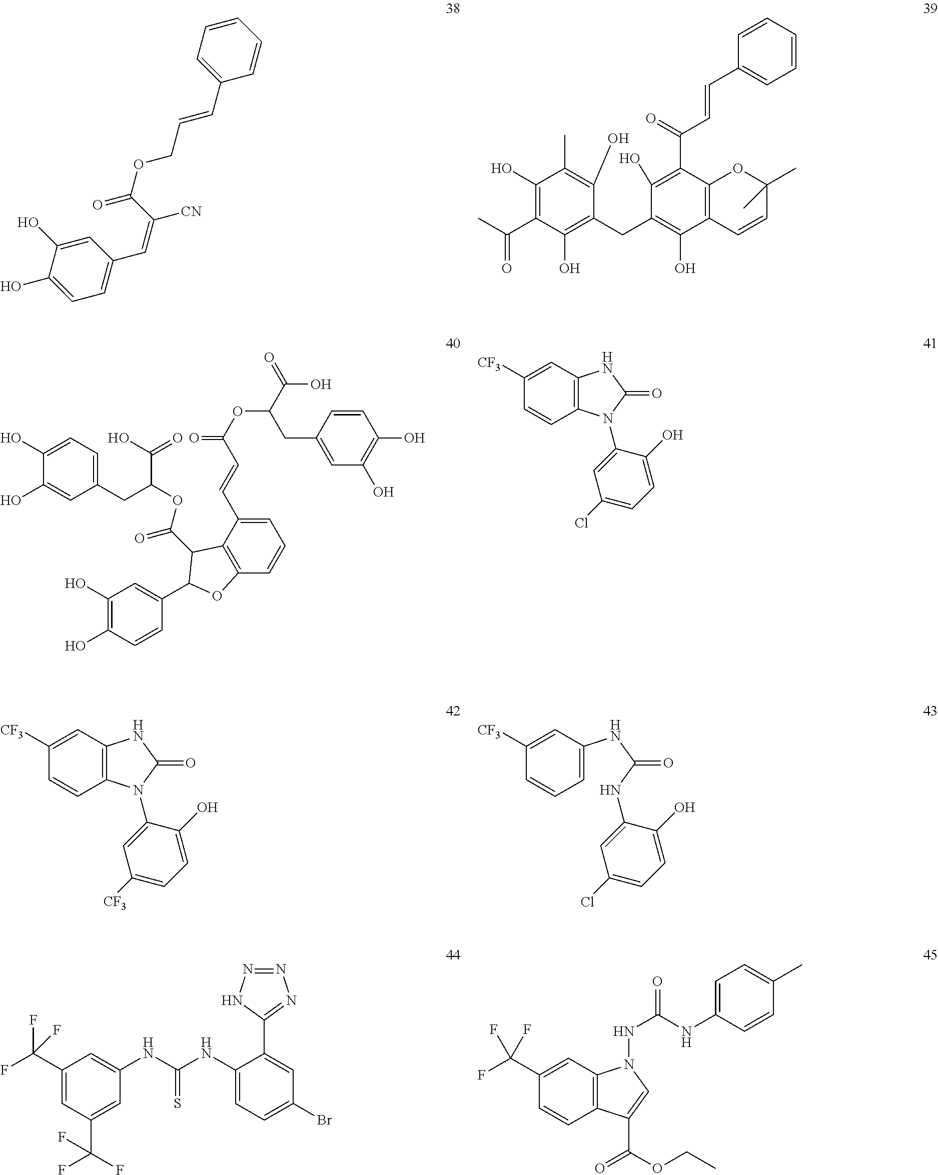 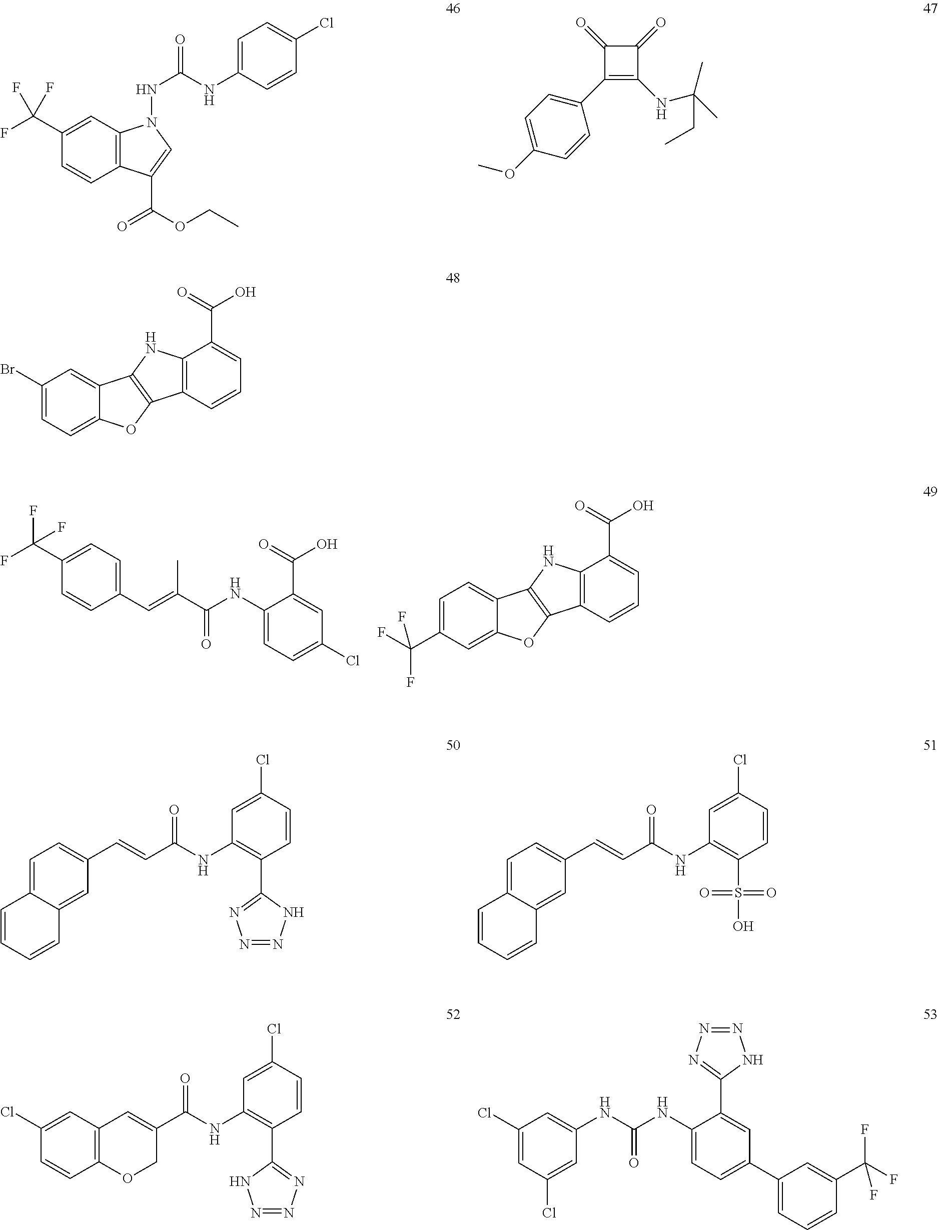 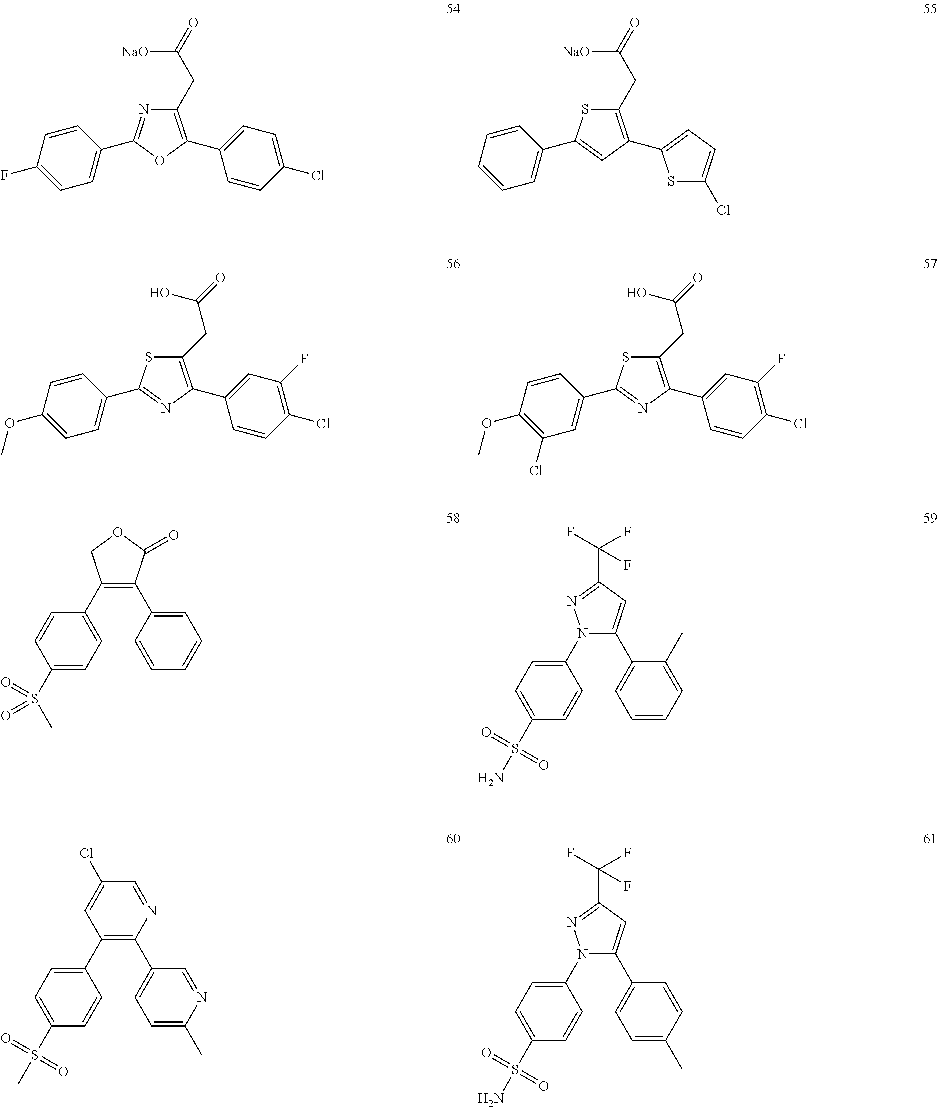 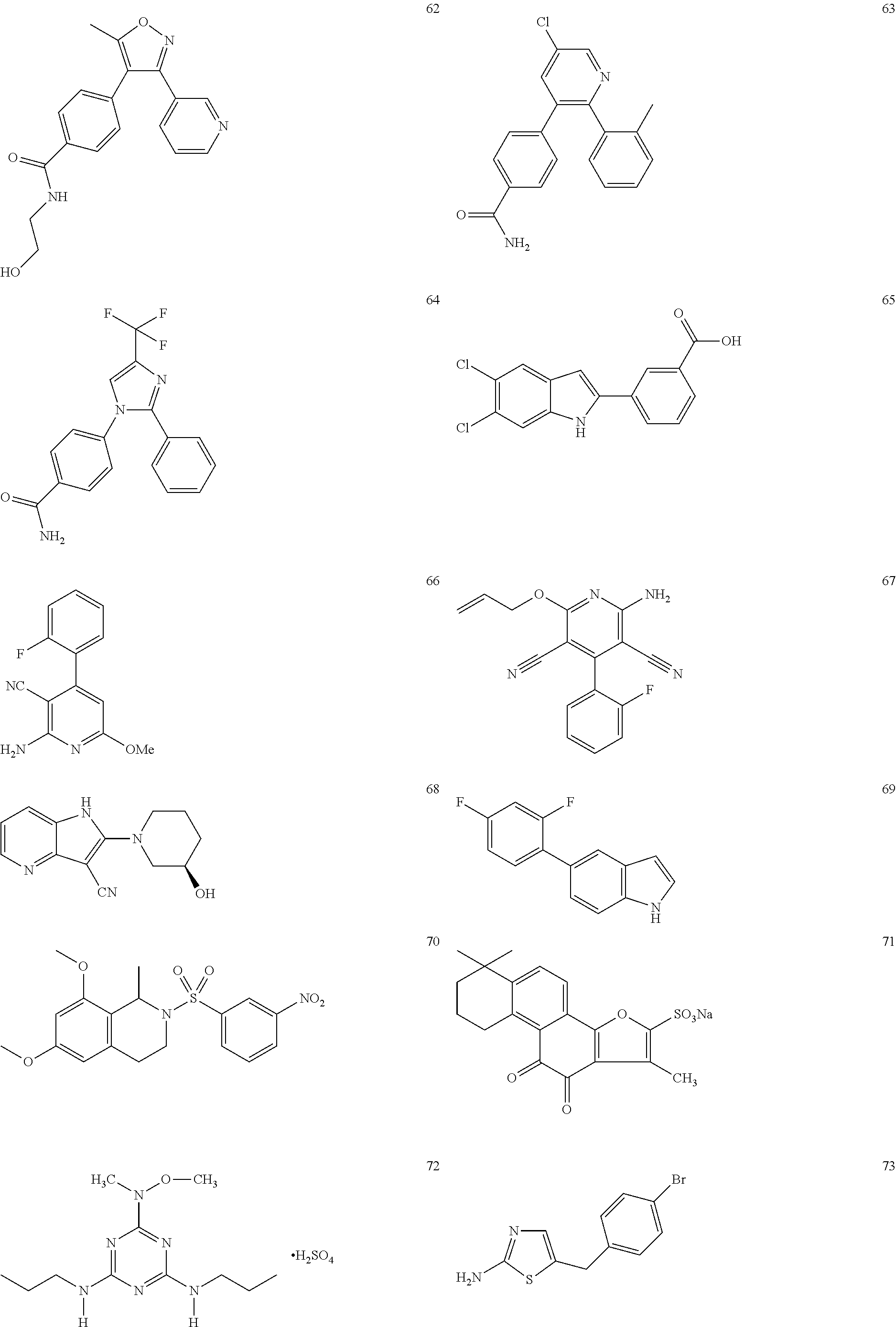
or LDD175 (4-chloro-7-trifluoromethyl-10Hbenzo[4,5]furo[3,2-b]indole-1-carboxylic acid);
or pharmaceutically acceptable salts, esters or hydrates thereof.

14-15. (canceled)

16. The method according to claim 13 wherein the BKCa channel activator is selected from:
TA1702 (Tanabe Seiyaku);

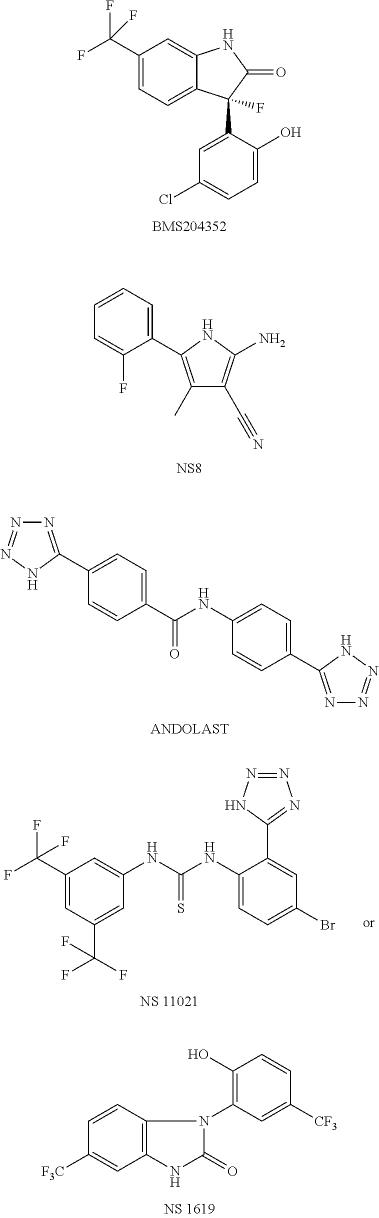

17. The method according to claim 13 wherein the BKCa channel activator is in admixture with a pharmaceutically acceptable diluent, excipient or carrier.
